(19) United States
(12) Patent Application Publication
(10) Pub. No.: US 2023/0183809 A1
(43) Pub. Date: Jun. 15, 2023

(54) EXTRACELLULAR VESICLES FOR TREATMENT AND DIAGNOSIS

(71) Applicant: EXOSTEM BIOTEC LTD., Tel Aviv (IL)

(72) Inventor: Aharon BRODIE, Meitar (IL)

(73) Assignee: EXOSTEM BIOTEC LTD., Tel Aviv (IL)

(21) Appl. No.: 17/920,858

(22) PCT Filed: Apr. 22, 2021
(86) PCT No.: PCT/IL2021/050466

§ 371 Date: Oct. 24, 2022

Related U.S. Application Data
(60) Provisional application No. 63/013,696, filed on Apr. 22, 2020.

(30) Foreign Application Priority Data
Mar. 11, 2021 (WO) PCT/IL2021/050274

(51) Int. Cl. C12Q 1/6886

(52) U.S. Cl. CPC C12Q 1/6886; C12Q 2600/158; C12Q 2600/178

(57) ABSTRACT

The present invention provides methods of detecting a cellular state in a subject, comprising providing a sample from the subject comprising extracellular vesicles (EVs), isolating a population of EVs originating from a tissue or cell type from the sample, and determining in the population of EVs the presence of at least one factor indicative of the cellular state, wherein the presence of the at least one factor indicates the cellular state is present in the tissue or cell type, thereby detecting a cellular state in a subject.

### CROSS REFERENCE TO RELATED APPLICATIONS

This application claims the benefit of priority of U.S. Provisional Pat. Application No. 63/013,696, filed on Apr. 22, 2020, and International Patent Application No. PCT/IL2021/050274, filed on Mar. 11, 2021, the contents of which are all incorporated herein by reference in their entirety.

### FIELD OF INVENTION

The present invention relates to diagnostic and treatment methods using extracellular vesicles (EVs).

### BACKGROUND OF THE INVENTION

Non-invasive diagnostic methods are of great interest as they allow for tailored treatment regimens without costly and unpleasant testing. Many such methods rely on circulating biomarkers such as cell-free DNA, circulating tumor cells, soluble proteins as well as circulating RNAs. Unfortunately, these methods have several drawbacks. These include a lack of reliability/reproducibility due to degradation and clearance from the blood and general short half-life of circulating factors. Moreover, it is not possible to correlate the circulating factor both to its cellular/tissue of origin and learn about the conditions present in that place of origin.

EVs are membrane-bound nanovesicles with diameters of 30-150 nm that contain multiple proteins, nucleic acid, lipids and other molecules in a tissue- and cell-specific manner. EVs are secreted by a large variety of cells. They play major roles in cell-cell interactions and in multiple physiological and pathological conditions. EVs have been demonstrated to exert therapeutic effects in various pathological conditions via the delivery of a diverse cargo including miRNAs, lncRNAs, DNA molecules, proteins and lipids. Methods of detecting different pathological conditions in a subject using circulating EVs is therefore greatly in need.

### SUMMARY OF THE INVENTION

The present invention provides methods of detecting a cellular state in a subject, comprising providing a sample from the subject comprising extracellular vesicles (EVs), isolating a population of EVs originating from a tissue or cell type from the sample, and determining in the population of EVs the presence of at least one factor indicative of the cellular state, wherein the presence of the at least one factor indicates the state is present in the tissue or cell type, thereby detecting a state in a subject.

According to a first aspect, there is provided a method of detecting an adverse cellular state in a subject in need thereof, the method comprising:
(i) providing a sample obtained from the subject, the sample comprising extracellular vesicles (EVs);
(ii) isolating from the sample a population of EVs originating from a tissue or cell type; and
(iii)determining in the population of EVs a presence of at least one factor indicative of the adverse cellular state;

wherein the presence of the at least one factor indicates the adverse cellular state is present in the tissue or cell type;
thereby detecting an adverse cellular state in a subject in need thereof.

According to some embodiments, the isolating comprises determining whether an EV of the population comprises a tissue, or cell-type specific marker; and a positive determination of a presence of the tissue, or cell-type specific marker is indicative of the origin of the EV.

According to some embodiments, the isolating comprises contacting the sample with an antibody or antigen binding fragment specific to a tissue or cell-type specific marker and isolating the antibody or antigen binding fragment.

According to some embodiments, the antibody or antigen binding fragment is conjugated to a solid support and the isolating comprises isolating the solid support.

According to some embodiments, the solid support is a magnetic bead.

According to some embodiments, the tissue or cell-type specific marker is a tissue or cell-type specific surface protein.

According to some embodiments, the tissue or cell-type specific surface protein is selected from Table 1.

According to some embodiments, the method of the invention further comprises before (ii) isolating total EVs from the sample and wherein the population of EVs is isolated from the total EVs.

According to some embodiments, the total EVs are isolated by contacting the sample with an antibody or antigen binding fragment specific to EVs and isolating the antibody or antigen binding fragment.

According to some embodiments, the antibody or antigen binding fragment specific to EVs a) is conjugated to a solid support, b) binds to a target selected from CD81, CD9 and CD63 or c) both.

According to some embodiments, the adverse cellular state is selected from an inflammatory state, a damaged state, a hypoxic state, a stressed state, a state of infection, an immunosuppressed state, a metastatic state and a hyper-proliferative state.

According to some embodiments, the at least one factor indicative of the adverse cellular state is selected from a protein and a regulatory RNA.

According to some embodiments, the regulatory RNA is selected from a microRNA (miR), a long noncoding RNA (lncRNA) and a circular RNA (circRNA).

According to some embodiments, the regulatory RNA is selected from Table 2.

According to some embodiments, the adverse cellular state is indicative of a pathological condition in the subject.

According to some embodiments, the subject suffers from a disease and the cellular state is indicative of disease activity in the tissue or cell-type, disease symptoms in the tissue or cell-type, disease origin from the tissue or cell-type or a combination thereof.

According to some embodiments, the method is a method of early detection of a disease or of a disease symptom.

According to some embodiments, the subject suffers from a disease and is receiving treatment for the disease and wherein the method is a method of determining responsiveness of the subject to the treatment.

According to some embodiments, a decrease in the adverse cellular state in the subject as compared to a time point before the treatment or earlier in the treatment indicates responsiveness to the treatment.

According to some embodiments, the method of the invention further comprises administering a treatment to the subject wherein the treatment is selected to treat the adverse cellular condition, to treat the tissue or cell type or both.

According to some embodiments, the treatment comprises therapeutic extracellular vesicles.

According to some embodiments, the therapeutic extracellular vesicles are selected from extracellular vesicles derived from any one of mesenchymal stromal cells (MSCs), plant cells and milk.

According to some embodiments, the extracellular vesicles are derived from chorionic placenta MSCs, umbilical cord MSCs or milk.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A bar graph of relative Newcastle Disease Virus (NDV) RNA detected in EVs from uninfected and infected THP-1 cells at various time points after infection. PCR levels at 48 hours were set as 1.

FIG. 2: A bar graph of relative mRNA expression of astrocyte, microglia and neuronal markers in EVs from neuronal cocultures, kidney cells and lung cells. Expression in the neuronal cocultures is standardized to 1. P<0.001

FIG. 3: A bar graph of relative expression of cytokines in PMA treated THP-1 cells, showing relative M1/M2 polarization. * = Pval <0.05, **=Pval<0.001, ****=Pval<0.0001.

FIG. 4: A bar graph of relative expression of cytokines in macrophages stimulated with LPS. *=Pval<0.002, **=Pval<0.001.

FIG. 5: A bar graph of the percent of cells that are fluorescent after additions of fluorescently labeled EVs.

FIG. 6: A bar graph of relative M2 polarization in human macrophages exposed to various cancer derived EVs with or without the addition of chorionic placenta (CH), umbilical cord (UC) or milk derived EVs.

### DETAILED DESCRIPTION OF THE INVENTION

The present invention, in some embodiments, provides methods of detecting a cellular state in a subject, comprising providing a sample from the subject comprising extracellular vesicles (EVs), isolating a population of EVs originating from a tissue or cell type from the sample, and determining in the population of EVs the presence of at least one factor indicative of the cellular state, thereby detecting a cellular state in a subject.

The content of EVs is impacted by various pathological conditions in the cells and reflects quite accurately the pathophysiology of the originating cells. Since the EV cargo is encapsulated, it is protected from degradation and therefore stable and consistent and can be accurately detected in body fluids. Therefore, changes in various diseases and pathological conditions can be identified and monitored by analyzing EV’s cargo. In addition to their cargo, the amount of secreted EVs and their characterization is also altered in various stress-related and pathological conditions and can serve as additional parameters to identify pathological conditions, albeit in a less specific manner.

The invention is based, at least in part, on the surprising finding that EVs can serve as circulating biomarkers for various diseases and as mediators of disease pathogenesis. It has been found that diseased or damaged cells secrete large quantities of EVs. These are a rich source of molecules that reflect the activity and pathophysiological state of the originating cells. Due to their selective molecular packaging and stability against degrading enzymes, secreted EVs represent a viable and consistent reservoir of biomarkers in human fluids. Therefore, they have emerged as attractive candidates for liquid biopsy.

The invention is further based on the finding that certain surface proteins are expressed on the surface of EVs and act there as tissue/cell-type specific markers of the origin of the EVs. These surface markers, it was surprisingly found, are not always the same markers found on the cells surface and indeed some tissue/cell-type specific markers known to reside on cells are not found on their EVs. These unique markers allow for the determination and isolation of pure populations of EVs from a given tissue or cell-type. These EVs can then be analyzed and provide information about the tissue/cell-type that produced them.

It was further found that specific factors within EVs are particularly informative about the cellular state present in the cells/tissues that produced the EVs. By analyzing these factors, the cellular state of particular organs/tissues/cells can be determined, and indeed cellular damage can be identified when no other signs are present. The fact that damaged/stressed cells overproduce EVs enhances the usefulness of this assay.

### Diagnosis of a Pathological Condition and Personalized Therapy

By a first aspect, there is provided a method of determining a pathological condition in a subject in need thereof, the method comprising:
(i) providing a sample obtained from the subject, said sample comprising an extracellular vesicle (EV);
(ii) providing at least one parameter of an EV associated with the pathological condition;
(iii) determining if the EV comprises the at least one parameter;
wherein a positive determination of the at least one parameter is indicative that the EV is a pathologically associated EV and of the pathological condition in the subject.

By another aspect, there is provided, a method of detecting a cellular state in a subject, the method comprising:
(i) providing a sample from the subject;
(ii) isolating a population of EVs from the sample;
(iii) and determining in the population of EVs a presence of at least one factor indicative of the cellular state;
thereby detecting a cellular state in a subject.

In some embodiments, providing a sample is obtaining a sample. In some embodiments, in some embodiments providing a sample is withdrawing a sample from the subject. In some embodiments, the method further comprises extracting the sample from the subject. In some embodiments, the sample is from the subject. In some embodiments, the sample is directly from the subject. In some embodiments, the sample is purified EVs from the subject.

In some embodiments, the sample is a biological sample. In some embodiments, the sample is a fluid sample. In some embodiments, the sample is a biopsy. In some embodiments, the sample is a skin sample. In some embodiments, the sample is a bodily fluid. In some embodiments, the bodily fluid is selected from: blood, serum, plasma, gastric fluid, intestinal fluid, saliva, bile, breast milk, nasal swab, oral swab, cerebral spinal fluid, urine, interstitial fluid, sperm, and stool. In some embodiments, the bodily fluid is blood. In some embodiments, the bodily fluid is serum. In some embodiments, the bodily fluid is plasma. In some embodiments, the blood is peripheral blood. In some embodiments, the bodily fluid is saliva. In some embodiments, the sample is from the subject. In some embodiments, the sample is a nasal swab. In some embodiments, the sample is an oral swab. In some embodiments, the sample is an anal swab. In some embodiments, the sample is a blood draw. In some embodiments, the sample comprises an EV. In some embodiments, the sample comprises EVs. In some embodiments, the sample is depleted of cells. In some embodiments, the sample is devoid of cells. In some embodiments, the sample is a not a tissue sample. In some embodiments, the sample is a liquid biopsy.

In some embodiments, the subject is a mammal. In some embodiments, the mammal is a human. In some embodiments, the subject suffers from a disease. In some embodiments, the subject suffers from a condition. In some embodiments, the subject is at risk for developing a disease or condition. In some embodiments, the subject is suspected of having a disease or condition. In some embodiments, the subject is confirmed to have a disease or condition. In some embodiments, the subject does not yet display symptoms of the disease or condition. In some embodiments, the subject is being treated for the disease or condition. In some embodiments, the subject has completed treatment for the disease or condition. In some embodiments, the subject is naive to treatment. In some embodiments, the subject has a disease or condition of unknown cause. In some embodiments, the subject is a subject in need of the detection of the method. In some embodiments, the subject is a subject in need of the method of the invention. In some embodiments, the subject is a subject in need thereof.

“Extracellular vesicles”, and “EVs” as used herein, are interchangeable and refer to cell-derived extracellular vesicles secreted from various cells including but not limited to exosomes and microvesicles. EVs refer to free floating vesicles and not vesicles that are matrix or membrane bound. “Exosome”, as used herein, refers to cell-derived extracellular vesicles of endocytic origin that are secreted from cells. In some embodiments, an exosome comprises a diameter of 30-150 nm. In some embodiments, an exosome comprises a diameter of 30-100 nm. In some embodiments, an exosome comprises a diameter of 50-100 nm. In some embodiments, an exosome comprises a diameter of 50-150 nm. “Microvesicles”, as used herein, refers to cell-derived extracellular vesicles originating from the plasma membrane, with a size of 30-1000 nm, and secreted from MSCs. Methods of isolating and purifying EVs are well known in the art. For example, EVs as a whole, can be isolated by differential centrifugation. In some embodiments, the extracellular vesicles are cell-derived vesicles. In some embodiments, the extracellular vesicles are not apoptotic bodies. In some embodiments, the EVs are matrix-bound vesicles (MBVs). In some embodiments, the EVs are not MBVs. “Matrix-bound vesicles” and “MBVs”, as used herein, are interchangeable and refer to extracellular vesicles that are secreted by various cells but are not soluble and free floating but rather bind to extracellular matrix (ECM) and are anchored there.

In some embodiments, the method further comprises determining whether the EV or EV population comprises a tissue, organ, or cell-originating marker; wherein a positive determination of the presence of the tissue, organ, or cell-originating marker is indicative of the origin of said EV. In some embodiments, the isolating is isolating from the sample. In some embodiments, the isolating is isolating a population of EVs originating from a tissue, organ or cell-type. In some embodiments, the EVs originate from a tissue. In some embodiments, the EVs originate from an organ. In some embodiments, the EVs originate from a cell type. In some embodiments, the tissue is a specific tissue. In some embodiments, the organ is a specific organ. In some embodiments, the cell-type is a specific cell-type. In some embodiments, the isolating comprises determining whether an EV of the population comprises a tissue, organ or cell-type specific marker. In some embodiments, the isolating comprises isolating a population of EVs comprising a tissue, organ or cell-type specific marker.

In some embodiments, the isolating comprises contacting the sample with a binding agent that specifically binds to the marker. In some embodiments, the marker is a tissue-specific marker. In some embodiments, the marker is an organ-specific marker. In some embodiments, the marker is a cell-type-specific marker. In some embodiments, the binding agent is a protein or protein fragment that binds the marker. In some embodiments, the binding agent is an antibody or antibody fragment that binds the marker. In some embodiments, the isolating comprises isolating the binding agent. In some embodiments, the isolating comprises isolating the binding agent bound to the marker.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi- specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv- Fc fusions, variable region (e.g., VL and VH)~ Fc fusions and scFv-scFv-Fc fusions.

Immunoglobulin molecules can be of any type (e.g., IgG, IgE, IgM, IgD, IgA and IgY), class (e.g., IgGl, IgG2, IgG3, IgG4, IgAl and IgA2) or subclass.

In some embodiments, the binding agent is conjugated to a support. The term “support” or “substrate,” as used herein, generally refers to any solid or semi-solid article on which reagents such as antibodies may be immobilized. In some embodiments, the support is a solid support. In some embodiments, the support is a substrate. In some embodiments, the support is a particle. In some embodiments, the particle is a bead. In some embodiments, the support is a column. In some embodiments, the support is an array. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations.

The term “solid support” refers to any artificial solid structure, including any solid support or substrate. Examples of solid supports include, but are not limited to, beads, resins, gels, hydrogels, colloids, particles, or nanoparticles. For example, a solid support may be a bead. Alternatively, the solid support may be a surface. For example, a solid support may comprise a bead coupled to a surface. Alternatively, the solid support may be a resin. The solid support may be isolatable. The solid support may be tagged. The solid support may be magnetic and isolatable with a magnet. Alternatively, or in addition to, the solid support may be isolated by centrifugation or some other force that separates by weight, size, or some other measurable quantity.

A support (e.g., a solid support) may be or comprise a particle. A particle may be a bead. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. In some embodiments, the bead is magnetic. For example, a bead may comprise one or more polymers bearing one or more magnetic labels. A magnetic bead may be manipulated (e.g., moved between locations or physically constrained to a given location, e.g., of a reaction vessel such as a flow cell chamber) using electromagnetic forces. A bead may have any useful shape, including, for example, a shape that is approximately cubic, spherical, ellipsoidal, dumbbell-shaped, or any other shape. For example, a bead may be approximately spherical in shape. A bead may have one or more different dimensions including a diameter. A dimension of the bead (e.g., a diameter of the bead) may be less than about 1 mm, less than about 0.1 mm, less than about 0.01 mm, less than about 0.005 mm, less than about 1 nm, less than about 1 µm, or smaller. A dimension of the bead (e.g., a diameter of the bead) may be between about 1 nm to about 100 nm, about 1 µm to about 100 µm, about 1 mm to about 100 mm. A collection of beads may comprise one or more beads having the same or different characteristics. For example, a first bead of a collection of beads may have a first diameter and a second bead of the collection of beads may have a second diameter. The first diameter may be the same or approximately the same as or different from the second diameter. Similarly, the first bead may have the same or a different shape and composition than a second bead. In some embodiments, the support is an artificial support. In some embodiments, the support is a manmade support. In some embodiments, the support is a magnetic bead. In some embodiments, the isolating is isolating the support. In some embodiments, the isolating is with a magnet or magnetic field.

In some embodiments, the marker is a protein. In some embodiments, the marker is a surface protein. In some embodiments, the marker is a surface receptor. In some embodiments, the marker is selected from a marker provided in Table 1. In some embodiments, the marker uniquely identifies the tissue, organ or cell-type. In some embodiments, the marker is a plurality of markers. In some embodiments, the marker is at least 1, 2, 3, 4, 5, 6, 7, 8, 9, or 10 markers. Each possibility represents a separate embodiment of the invention. In some embodiments, the marker is a sufficient number of markers to uniquely identify the origin of the EV population. In some embodiments, the marker is a sufficient number of markers to identify a unique EV population by its origin.

In some embodiments, the tissue or organ is selected from the brain, the heart, the lungs, the liver and the kidneys. In some embodiments, the tissue or organ is the brain. In some embodiments, the tissue or organ is the heart. In some embodiments, the tissue or organ is the lungs. In some embodiments, the tissue or organ is the liver. In some embodiments, the tissue or organ is the kidneys. Examples of tissues and organs are well known in the art and include for examples, the brain, the heart, the lungs, the liver, the larynx, the throat, the tongue, the glands, the pancreas, the skin, the gallbladder, the stomach, the small intestine, the large intestine, the bladder, the kidneys, the lymph nodes, the testis, the ovary, the uterus, the muscle, the fat, the blood vessels, the bone, and the bone marrow to name but a few. Tissue and organ specific markers can be determined as described hereinbelow. In some embodiments, the tissue or organ is a tissue or organ or an embryo or fetus. In some embodiments, the tissue or organ is an embryonic or fetal tissue or organ.

In some embodiments, the cell-type is from within a tissue. In some embodiments, the cell-type is a brain cell type. In some embodiments, the cell type is selected from astrocytes, microglia, oligodendrocytes, meninges, cerebral blood vessel cells and neurons. In some embodiments, the cell type is selected from astrocytes, microglia neurons. In some embodiments, the cell type is astrocytes. In some embodiments, the cell type is microglia. In some embodiments, the cell type is neurons. In some embodiments, neurons are mature neurons. In some embodiments, the cell type is glial cells. In some embodiments, the cell type is an immune cell type. In some embodiments, the immune cell type is selected from B cells, T cells, NK cells and dendritic cells. In some embodiments, the T cells are selected from CD4 T cells and CD8 T cells. In some embodiments, the cell type is muscle cell types. In some embodiments, muscle cells are selected from smooth muscle cells and rough muscle cells. Cell types are well known in the art, and but a few are recited here. Any cell type and cell type specific markers may be used. In some embodiments, the cell-type is an embryonic or fetal cell type.

In some embodiments, the tissue or organ is brain and the marker is selected from ADAM11, AKAP5, APLP1, ASIC2, ATP2B3, BCAN, CACNG8, CASKIN1, CNTN2, CNTNAP2, DLG4, DRD2, DRD5, ERC2, GABRA1, GABRA3, GABRB2, GPR158, GPR26, GRIA2, GRIK4, GRM2, GRM4, HCRTR2, HS3ST5, HTR6, KCNA1, KCNA2, KCNA3, KCNF1, KCNG2, KCNJ3, KCNJ9, KCNK13, LEPR, MC3R, MLC1, MYO15A, NCAN, NPTXR, NRSN1, NRXN3, PACSIN1, PDYN, PIEZO1, PLXNB3, PRL, PTPN5, PTPRZ1, RGS7, SCN2A, SLC12A5, SLC1A2, SLC1A3, SLC6A11, SLC6A17, SLC8A2, SLITRK1, SNRPN, SNTG1, SYT1, TAGLN3, TMEM59L, and TRHR.

In some embodiments, the cell type is astrocytes and the marker is selected from PDYN, SNRPN, TRHR, ASIC2, SNTG1, GRM2, PTPN5, NRXN3, PTPRZ1, SLC1A2, RGS7, AQP4, and SLC1A2.

In some embodiments, the cell type is microglia and the marker is selected from TMEM119, CR3, CSF1R, Siglec-H, CX3CR1, CD200, P2yR12, Tyro3, AXL, Mertk, Cd11c, CD68, CD11b, CD80, CD115, CD16, FCER1G, CD16, CA115, EMR1, TREM2, RCA1, Glut5, and CD32.

In some embodiments, the cell type is neurons and the marker is selected from Kv2.2, Kv2.1, Kv4.3, Nav1.4, SLC1A3, N-Cadherin, NG-2, synaptophysin, PSD95, vGluT1, vGlut2, NMDR1, NMDAR2B, GAT1, GABAb receptor 1, GAGAb receptor 2, DAT, CD15, CD24, and CD29.

In some embodiments, the tissue or organ is brain and the marker is selected from Kv2.2, Kv2.1, Kv4.3, Nav1.4, SLC1A3, N-Cadherin, NG-2, synaptophysin, PSD95, vGluT1, vGlut2, NMDR1, NMDAR2B, GAT1, GABAb receptor 1, GAGAb receptor 2, DAT, CD15, CD24, and CD29. In some embodiments, the tissue or organ is brain and the marker is selected from ADAM11, AKAP5, APLP1, ASIC2, ATP2B3, BCAN, CACNG8, CASKIN1, CNTN2, CNTNAP2, DLG4, DRD2, DRD5, ERC2, GABRA1, GABRA3, GABRB2, GPR158, GPR26, GRIA2, GRIK4, GRM2, GRM4, HCRTR2, HS3ST5, HTR6, KCNA1, KCNA2, KCNA3, KCNF1, KCNG2, KCNJ3, KCNJ9, KCNK13, LEPR, MC3R, MLC1, MYO15A, NCAN, NPTXR, NRSN1, NRXN3, PACSIN1, PDYN, PIEZO1, PLXNB3, PRL, PTPN5, PTPRZ1, RGS7, SCN2A, SLC12A5, SLC1A2, SLC1A3, SLC6A11, SLC6A17, SLC8A2, SLITRK1, SNRPN, SNTG1, SYT1, TAGLN3, TMEM59L, TRHR, Kv2.2, Kv2.1, Kv4.3, Nav1.4, SLC1A3, N-Cadherin, NG-2, synaptophysin, PSD95, vGluT1, vGlut2, NMDR1, NMDAR2B, GAT1, GABAb receptor 1, GAGAb receptor 2, DAT, CD15, CD24, and CD29.

In some embodiments, the tissue or organ is heart and the marker is selected from MYL4 and NPPA, Kir2.1, HCN4, epsilon Sarcoglycan, CD77, CD326, SIRPA, or Troponin T.

In some embodiments, the tissue or organ is kidney and the marker is selected from AQP6, ATP6V1G3, CLDN16, FGB, NPHS1, NPHS2, SLC22A12, SLC22A13, SLC22A2, SLC22A6, SLC22A8, SLC34A1, SLC34A3, SLC4A1, and UMOD.

In some embodiments, the tissue or organ is liver and the marker is selected from ABCB4, ASGR2, C4B, CPB2, F2, HP, HPR, IGFALS, LPA, MAT1A, MBL2, PROZ, SLC10A1, SLC13A5, SLC27A5, SLCO1B1, SLCO1B3, and UROC1.

In some embodiments, the tissue or organ is liver and the marker is selected from ACP5, SCGB1A1 and SFTTPB.

In some embodiments, the method further comprises isolating total EVs. In some embodiments, the total EVs are isolated from the sample. In some embodiments, the total EVs are isolated before step (ii). In some embodiments, the population of EVs is isolated from the total EVs. In some embodiments, the EV is selected from the total EVs. In some embodiments, total EVs are isolated by centrifugation. In some embodiments, centrifugation is differential centrifugation. In some embodiments, the total EVs are isolated by contacting the sample with a binding agent that binds EVs. In some embodiments, the binding agent is a pan-EV binding agent. In some embodiments, the binding agent is specific to EVs. In some embodiments, the pan-EV binding agent does not bind cells. In some embodiments, the binding agent specific to EVs binds to a marker specific to EVs. In some embodiments, the marker specific to EVs is a surface protein specific to EVs. In some embodiments, the surface protein is selected from CD81, CD9 and CD63. In some embodiments, the marker for EVs is CD81. In some embodiments, the marker for EVs is CD9. In some embodiments, the marker for EVs is CD63.

It will be understood by a skilled artisan that the tissue/organ/cell-type specific markers may also bind to the cell of origin and not just the EVs. As there may be cells in the sample and the assay desires a population of EVs that is devoid or depleted of cells, the isolation first of EVs using EV specific markers removes an cells that may be present and then the tissue/organ/cell-type specific cannot also pull-down undesired cells or cellular fragments.

In some embodiments, the presence of a factor is determined in an EV. In some embodiments, the presence of a factor is determined in a population of EVs. In some embodiments, the presence of a factor is determined in a population of EVs from a tissue, organ or cell-type. In some embodiments, the presence of the at least one factor indicates the cellular state is present. In some embodiments, the presence of the at least one factor indicates the cellular state is present in the tissue, organ or cell-type. In some embodiments, the presence of the at least one factor indicates the cellular state is present in the tissue, organ or cell-type from which the EV population originated. In some embodiments, the factor is a marker of the cellular state.

In some embodiments, the factor is at least one factor. In some embodiments, the factor is a plurality of factors. In some embodiments, the factor is at least 1, 2, 3, 4, 5, 6, 7, 8, 9, or 10 factors. Each possibility represents a separate embodiment of the invention. In some embodiments, the factor is indicative of a general cellular state. In some embodiments, the factor is indicative of a cellular state in a specific tissue, organ or cell-type. In some embodiments, the plurality of factors comprises at least one factor that is indicative of a general cellular state and at least one factor that is indicative of a cellular state in a specific tissue, organ or cell-type.

In some embodiments, the factor is selected from a protein, and an RNA. In some embodiments, the factor is a protein. In some embodiments, the factor is an RNA. In some embodiments, the RNA is an mRNA. In some embodiments, the RNA is a regulatory RNA. In some embodiments, the regulatory RNA is selected from a microRNA (miR), a long noncoding RNA (IncRNA) and a circular RNA (circRNA). In some embodiments, the regulatory RNA is a miR. In some embodiments, the regulatory RNA is a IncRNA. In some embodiments, the regulatory RNA is circRNA. In some embodiments, the factor is selected from Table 2. In some embodiments, the factor is selected from the factors provided in Table 2. In some embodiments, the regulatory RNA is selected from Table 2. In some embodiments, the regulatory RNA is selected from the regulatory RNAs provided in Table 2. In some embodiments, the protein is selected from Table 2. In some embodiments, the protein is selected from the proteins provided in Table 2.

In some embodiments, a cellular state is a condition. In some embodiments, a cellular state is an adverse cellular state. In some embodiments, the cellular state is a disease state. In some embodiments, the cellular state is pathological state. In some embodiments, the cellular state is a pathophysiological state. In some embodiments, the cellular state is an injured state. In some embodiments, the cellular state is a damaged state. In some embodiments, the cellular state is a non-healthy cellular state. In some embodiments, the cellular state is an abnormal cellular state. In some embodiments, the cellular state is a non-optimal cellular state. In some embodiments, the cellular state is not the natural state of the cell. In some embodiments, the cellular state is a stressed cellular state.

In some embodiments, the cellular state is an inflammatory state. In some embodiments, the cellular state is a damaged state. In some embodiments, the cellular state is a hypoxic state. In some embodiments, the cellular state is stressed state. In some embodiments, stressed is ER stress. In some embodiments, stress is mitochondrial stress. In some embodiments, the cellular state is a state of infection. In some embodiments, the infection is viral infection. In some embodiments, the infection is bacterial infection. In some embodiments, the cellular state is an immunosuppressed state. In some embodiments, the cellular state is a metastatic state. In some embodiments, the cellular state is a hyperproliferative state.

In some embodiments, the cellular state is indicative of a pathological condition in the subject. In some embodiments, the cellular state is indicative of the development of symptoms associated with a pathological condition. In some embodiments, a pathological condition is cellular damage. In some embodiments, a pathological condition is tissue or organ damage. In some embodiments, the damage is brain damage. In some embodiments, the pathological condition is a disease. In some embodiments, the cellular state is indicative of a spread of the pathological condition. In some embodiments, the disease is an infection. In some embodiments, the infection is a bacterial infection. In some embodiments, the infection is a viral infection. In some embodiments, the disease is an inflammatory disease. In some embodiments, the disease is an autoimmune disease. In some embodiments, the disease is a hyperproliferative disease. In some embodiments, the disease is cancer. In some embodiments, the disease is metastasis of cancer. In some embodiments, the disease is sepsis. In some embodiments, the disease is a systemic disease. In some embodiments, the disease is a localized disease. In some embodiments, the disease is a fibrotic disease. In some embodiments, the disease is fibrosis. In some embodiments, the disease is characterized by fibrosis. In some embodiments, the cellular state is fibrosis. In some embodiments, the cellular state is a fibrotic state. In some embodiments, the disease is a degenerative disease. In some embodiments, the degenerative disease is a neurodegenerative disease. In some embodiments, the disease is a brain disease. In some embodiments, the disease is a stroke. In some embodiments, the disease is a treatable disease. In some embodiments, the infection is COVID-19. In some embodiments, the infection is a coronavirus infection. In some embodiments, the coronavirus is a beta coronavirus. In some embodiments, the disease is a psychiatric disease or disorder. In some embodiments, the disease is major depressive disorder. In some embodiments, the disease is heart disease. In some embodiments, the disease is coronary artery disease. In some embodiments, the disease is cardiomyopathy. In some embodiments, heart damage is cardiomyopathy. In some embodiments, a symptom of the disease is cardiomyopathy. In some embodiments, the disease is kidney disease. In some embodiments, the fibrosis is renal fibrosis. In some embodiments, the fibrosis is lung fibrosis. In some embodiments, the fibrosis is cystic fibrosis. In some embodiments, the disease is cystic fibrosis. In some embodiments, the fibrosis is liver fibrosis. In some embodiments, the disease is fatty liver disease. In some embodiments, the disease is cellular damage. In some embodiments, the cellular damage is injury. In some embodiments, the damage is radiation induced damage. In some embodiments, the damage is therapeutic induced damage. In some embodiments, the damage is anesthetic induced damage. In some embodiments, the disease is a disease or an embryo or fetus. In some embodiments, the disease is a genetic disease. In some embodiments, the disease is a congenital disease. In some embodiments, the damage is damage to the fetus or embryo.

In some embodiments, cellular state is fibrosis and the factor is selected from CFTR, hsa_circ_0013255, hsa_circ_0054345, hsa_circ_0061893, hsa_circ_0070963, hsa_circ_0071410, hsa_circ_0071410, hsa_circ_0072765, PALLD, BGas, CDKN2B-AS1, H19, MEG3, uc.77, WT1-AS, hsa-mir-19a, hsa-mir-29, hsa-mir-18a, hsa-mir-155, hsa-mir-21, hsa-mir-214, hsa-mir-29b, hsa-mir-196a, hsa-mir-196b, hsa-mir-214, hsa-mir-19b, hsa-mir-19a, hsa-mir-19b, hsa-mir-26b, hsa-mir-29a, hsa-mir-145, hsa-mir-449a, hsa-mir-185, hsa-mir-186, hsa-mir-29a, hsa-mir-221, hsa-mir-208a, hsa-mir-25, hsa-mir-29a, hsa-mir-21, hsa-mir-23b, hsa-mir-27b, hsa-mir-30b, hsa-mir-21, hsa-mir-382, hsa-mir-155, hsa-mir-29a, hsa-mir-29b-1, hsa-mir-29b-2, hsa-mir-29c, hsa-mir-21, hsa-mir-21, hsa-mir-21, hsa-mir-31, hsa-mir-101-1, hsa-mir-101-2, hsa-mir-21, hsa-mir-33a, hsa-mir-214, hsa-mir-218, hsa-mir-509, hsa-mir-26a, hsa-mir-106b, hsa-mir-215, hsa-mir-199a, hsa-let-7e, hsa-mir-125a, hsa-mir-99b, hsa-mir-21, hsa-mir-29c, hsa-mir-153, hsa-mir-200, hsa-mir-486, hsa-mir-21, hsa-let-7d, hsa-mir-34a, hsa-mir-146a, hsa-mir-155, hsa-mir-370, hsa-mir-708, hsa-mir-378, hsa-mir-34a, hsa-mir-200c, hsa-mir-34a, hsa-let-7d, hsa-mir-154, hsa-mir-24, hsa-mir-18a, hsa-mir-29c, hsa-mir-181b, hsa-mir-23a, hsa-mir-27a, hsa-mir-199, hsa-mir-135a, hsa-mir-200b, hsa-mir-21, hsa-mir-410, hsa-mir-142, hsa-mir-222, hsa-mir-21, hsa-mir-200c, hsa-mir-196a, hsa-mir-30, hsa-mir-30e, hsa-mir-122, hsa-mir-326, hsa-mir-101, hsa-mir-92a, hsa-mir-29b, hsa-mir-203, hsa-mir-144, hsa-mir-193, hsa-mir-30, hsa-mir-130a, hsa-mir-27b, hsa-mir-1908, hsa-mir-21, hsa-mir-130b, hsa-mir-21, hsa-mir-134, hsa-mir-155, hsa-mir-9, hsa-mir-29c, hsa-mir-21, hsa-mir-18a, hsa-mir-98, hsa-mir-30a, hsa-mir-21, hsa-mir-29b, hsa-mir-30a, hsa-mir-152, hsa-mir-26a, hsa-mir-146a, hsa-mir-34a, hsa-mir-29b, hsa-mir-30d, hsa-mir-942, hsa-mir-7, hsa-mir-21, hsa-mir-200a, hsa-mir-542, hsa-mir-200, hsa-mir-454, hsa-mir-31, hsa-mir-133, hsa-mir-29, hsa-mir-21, hsa-mir-17, hsa-mir-16, hsa-mir-17, hsa-mir-27b, hsa-mir-21, hsa-mir-1343, hsa-mir-125b, hsa-mir-142, hsa-mir-21, hsa-mir-221, hsa-mir-223, hsa-mir-34a, hsa-mir-433, hsa-mir-130a, hsa-mir-29a, hsa-let-7d, hsa-mir-192, hsa-mir-26a, hsa-mir-210, hsa-mir-214, hsa-mir-140, hsa-mir-21, hsa-mir-133a, hsa-mir-30a, hsa-mir-18a, hsa-mir-212, hsa-mir-203, hsa-mir-221, hsa-mir-222, hsa-mir-21, hsa-mir-149, hsa-mir-155, hsa-mir-34, hsa-mir-34, and hsa-mir-34a. In some embodiments, cellular state is fibrosis and the factor is the protein or mRNA encoding CFTR. In some embodiments, cellular state is fibrosis and the factor is a circRNA selected from hsa_circ_0013255, hsa_circ_0054345, hsa_circ_0061893, hsa_circ_0070963, hsa_circ_0071410, hsa_circ_0071410, hsa_circ_0072765, PALLD. In some embodiments, cellular state is fibrosis and the factor is a lncRNA selected from BGas, CDKN2B-AS1, H19, MEG3, uc.77, WT1-AS. In some embodiments, cellular state is fibrosis and the factor is a miR selected from hsa-mir-19a, hsa-mir-29, hsa-mir-18a, hsa-mir-155, hsa-mir-21, hsa-mir-214, hsa-mir-29b, hsa-mir-196a, hsa-mir-196b, hsa-mir-214, hsa-mir-19b, hsa-mir-19a, hsa-mir-19b, hsa-mir-26b, hsa-mir-29a, hsa-mir-145, hsa-mir-449a, hsa-mir-185, hsa-mir-186, hsa-mir-29a, hsa-mir-221, hsa-mir-208a, hsa-mir-25, hsa-mir-29a, hsa-mir-21, hsa-mir-23b, hsa-mir-27b, hsa-mir-30b, hsa-mir-21, hsa-mir-382, hsa-mir-155, hsa-mir-29a, hsa-mir-29b-1, hsa-mir-29b-2, hsa-mir-29c, hsa-mir-21, hsa-mir-21, hsa-mir-21, hsa-mir-31, hsa-mir-101-1, hsa-mir-101-2, hsa-mir-21, hsa-mir-33a, hsa-mir-214, hsa-mir-218, hsa-mir-509, hsa-mir-26a, hsa-mir-106b, hsa-mir-215, hsa-mir-199a, hsa-let-7e, hsa-mir-125a, hsa-mir-99b, hsa-mir-21, hsa-mir-29c, hsa-mir-153, hsa-mir-200, hsa-mir-486, hsa-mir-21, hsa-let-7d, hsa-mir-34a, hsa-mir-146a, hsa-mir-155, hsa-mir-370, hsa-mir-708, hsa-mir-378, hsa-mir-34a, hsa-mir-200c, hsa-mir-34a, hsa-let-7d, hsa-mir-154, hsa-mir-24, hsa-mir-18a, hsa-mir-29c, hsa-mir-181b, hsa-mir-23a, hsa-mir-27a, hsa-mir-199, hsa-mir-135a, hsa-mir-200b, hsa-mir-21, hsa-mir-410, hsa-mir-142, hsa-mir-222, hsa-mir-21, hsa-mir-200c, hsa-mir-196a, hsa-mir-30, hsa-mir-30e, hsa-mir-122, hsa-mir-326, hsa-mir-101, hsa-mir-92a, hsa-mir-29b, hsa-mir-203, hsa-mir-144, hsa-mir-193, hsa-mir-30, hsa-mir-130a, hsa-mir-27b, hsa-mir-1908, hsa-mir-21, hsa-mir-130b, hsa-mir-21, hsa-mir-134, hsa-mir-155, hsa-mir-9, hsa-mir-29c, hsa-mir-21, hsa-mir-18a, hsa-mir-98, hsa-mir-30a, hsa-mir-21, hsa-mir-29b, hsa-mir-30a, hsa-mir-152, hsa-mir-26a, hsa-mir-146a, hsa-mir-34a, hsa-mir-29b, hsa-mir-30d, hsa-mir-942, hsa-mir-7, hsa-mir-21, hsa-mir-200a, hsa-mir-542, hsa-mir-200, hsa-mir-454, hsa-mir-31, hsa-mir-133, hsa-mir-29, hsa-mir-21, hsa-mir-17, hsa-mir-16, hsa-mir-17, hsa-mir-27b, hsa-mir-21, hsa-mir-1343, hsa-mir-125b, hsa-mir-142, hsa-mir-21, hsa-mir-221, hsa-mir-223, hsa-mir-34a, hsa-mir-433, hsa-mir-130a, hsa-mir-29a, hsa-let-7d, hsa-mir-192, hsa-mir-26a, hsa-mir-210, hsa-mir-214, hsa-mir-140, hsa-mir-21, hsa-mir-133a, hsa-mir-30a, hsa-mir-18a, hsa-mir-212, hsa-mir-203, hsa-mir-221, hsa-mir-222, hsa-mir-21, hsa-mir-149, hsa-mir-155, hsa-mir-34, hsa-mir-34, and hsa-mir-34a.

In some embodiments, cellular state is inflammation and the factor is selected from STAT3, hsa-mir-3560, hsa-mir-3588, hsa-mir-466b, hsa-mir-6315, hsa-mir-4661, hsa-mir-1270, hsa-mir-710, hsa-mir-155, hsa-mir-16, hsa-mir-21, hsa-mir-223, hsa-mir-106a, hsa-mir-362, hsa-mir-320, hsa-mir-505, hsa-mir-132, hsa-mir-212, hsa-mir-105, hsa-mir-132, hsa-mir-135a, hsa-mir-142, hsa-mir-147, hsa-mir-155, hsa-mir-223, hsa-mir-298, hsa-mir-299a, hsa-mir-325, hsa-mir-410, hsa-mir-181b-1, hsa-mir-21, hsa-mir-219, hsa-mir-181a, hsa-mir-181b, hsa-mir-21, hsa-mir-132, hsa-mir-221, hsa-mir-146b, hsa-mir-221, hsa-mir-98, hsa-mir-9, hsa-mir-223, hsa-mir-181a, hsa-mir-203, hsa-mir-146b, hsa-mir-132, hsa-mir-132, hsa-mir-221, hsa-mir-27b, hsa-mir-146a, hsa-mir-210, hsa-mir-146a, hsa-mir-125b, hsa-mir-221, hsa-mir-579, hsa-mir-155, hsa-mir-24-1, hsa-mir-24-2, hsa-mir-33a, hsa-mir-33b, hsa-mir-1, hsa-mir-16, hsa-mir-206, hsa-mir-210, hsa-mir-132, hsa-mir-212, hsa-mir-146a, hsa-mir-147, hsa-mir-126, hsa-mir-125b, hsa-mir-219-2, hsa-mir-181a-2, hsa-mir-21, hsa-mir-155, hsa-mir-21, hsa-mir-146a, hsa-mir-146, hsa-mir-21, hsa-mir-21, hsa-mir-93, hsa-mir-155, hsa-mir-146a, hsa-mir-34a, hsa-mir-126, hsa-mir-33a, hsa-mir-148a, hsa-mir-31, hsa-mir-146a, hsa-mir-17, hsa-mir-18, hsa-mir-19a, hsa-mir-19b, hsa-mir-19b-1, hsa-mir-20a, hsa-mir-449c, hsa-mir-455, hsa-mir-92-1, hsa-mir-223, hsa-mir-29a, hsa-mir-34a, hsa-mir-424, hsa-mir-129, hsa-mir-186, hsa-mir-200c, hsa-mir-222, hsa-mir-101, hsa-mir-22, hsa-mir-100, hsa-mir-122, hsa-mir-143, hsa-mir-125b, hsa-mir-146a, hsa-mir-146a, hsa-mir-132, hsa-mir-221, hsa-mir-146a, hsa-mir-181, hsa-mir-21, hsa-mir-98, hsa-mir-155, hsa-mir-210, hsa-mir-146a, hsa-mir-223, hsa-mir-182, hsa-mir-183, hsa-mir-133a, hsa-mir-155, hsa-mir-210, hsa-mir-132, hsa-mir-608, hsa-let-7, hsa-mir-124, hsa-mir-146a, hsa-mir-155, hsa-mir-21, hsa-mir-223, hsa-mir-27b, hsa-mir-326, hsa-mir-155, hsa-mir-125b, hsa-mir-223, hsa-mir-181a, hsa-mir-146, hsa-mir-155, hsa-mir-34a, hsa-mir-132, hsa-mir-146b, hsa-mir-155, hsa-mir-21, hsa-mir-221, hsa-mir-223, hsa-mir-128, hsa-mir-320, hsa-mir-350, hsa-mir-34c, hsa-mir-223, hsa-mir-192, hsa-mir-105, hsa-mir-25, hsa-mir-17, hsa-mir-31, hsa-mir-135a, hsa-mir-181a, hsa-mir-181b, hsa-mir-199b, hsa-mir-204, hsa-mir-9, hsa-mir-146b, hsa-mir-219, hsa-mir-29a, hsa-mir-155, hsa-mir-221, hsa-mir-222, hsa-mir-150, hsa-let-7a-1, hsa-let-7a-2, hsa-let-7a-3, hsa-let-7b, hsa-let-7c, hsa-let-7d, hsa-let-7e, hsa-let-7f-1, hsa-let-7f-2, hsa-let-7g, hsa-let-7i, hsa-mir-21, hsa-mir-1236, hsa-mir-127, hsa-mir-181b, hsa-mir-23b, hsa-mir-203, hsa-mir-203, hsa-mir-224, hsa-mir-181a, hsa-mir-9, hsa-mir-146b, hsa-mir-182, hsa-mir-146, hsa-mir-149, hsa-mir-224, hsa-mir-708, hsa-mir-155, hsa-mir-142, hsa-mir-24, hsa-mir-30b, hsa-mir-125b, hsa-mir-148a, hsa-mir-125b, hsa-mir-132, hsa-mir-143, hsa-mir-148a, hsa-mir-193b, hsa-mir-223, hsa-mir-221, hsa-mir-146b, hsa-mir-221, hsa-mir-155, hsa-mir-193a, hsa-mir-194, hsa-mir-744, hsa-mir-130a, hsa-mir-142, hsa-mir-150, hsa-mir-320, hsa-mir-328, hsa-mir-16, hsa-mir-125a, hsa-mir-124, hsa-mir-200b, hsa-mir-125b, hsa-mir-16, hsa-mir-191a, hsa-mir-142, hsa-mir-223, hsa-mir-146a, hsa-mir-203, hsa-mir-146a, hsa-mir-26a, hsa-mir-132, hsa-mir-29b, hsa-mir-15a, hsa-mir-155, hsa-mir-155, hsa-mir-143, hsa-mir-124, hsa-mir-133, hsa-mir-1246, hsa-mir-595, hsa-mir-146a, hsa-mir-142, hsa-mir-122, hsa-mir-21, hsa-mir-155, hsa-mir-146a, hsa-mir-1-1, hsa-mir-1-2, hsa-mir-133a, hsa-mir-133b, hsa-mir-206, hsa-mir-146a, hsa-mir-455, hsa-mir-155, hsa-mir-146a, hsa-mir-155, hsa-mir-155, hsa-mir-223, hsa-let-7i, hsa-mir-145, hsa-mir-346, hsa-mir-10a, hsa-mir-155, hsa-mir-125b, hsa-mir-200, hsa-let-7e, hsa-mir-338, hsa-mir-92b, hsa-mir-99a, hsa-mir-99b, hsa-mir-146a, hsa-mir-146b, hsa-mir-146, hsa-mir-15b, hsa-mir-181b, hsa-mir-31, hsa-mir-106a, hsa-mir-19b-1, hsa-mir-19b-2, hsa-mir-629, hsa-mir-146a, hsa-mir-206, hsa-mir-31, hsa-mir-424, hsa-mir-21, hsa-mir-132, hsa-mir-31, hsa-mir-99b, hsa-mir-223, hsa-mir-143, hsa-mir-150, hsa-mir-155, hsa-mir-17, hsa-mir-18, hsa-mir-181, hsa-mir-19a, hsa-mir-19b-1, hsa-mir-20a, hsa-mir-223, hsa-mir-92-1, hsa-mir-150, hsa-mir-584, hsa-mir-766, hsa-mir-132, hsa-mir-130b, hsa-mir-132, hsa-mir-21, hsa-mir-34a, hsa-mir-132, hsa-mir-221, hsa-mir-222, hsa-mir-146a, hsa-mir-132, hsa-mir-223, hsa-let-7b, CBR3-AS1, CBR3-AS1, GAS5, GAS5, NRON, circHECW2, HIPK2, and hsa-circRNA-2149. In some embodiments, the cellular state is inflammation and the factor is the protein or mRNA encoding STAT3. In some embodiments, the cellular state is inflammation and the factor is a miR selected from hsa-mir-3560, hsa-mir-3588, hsa-mir-466b, hsa-mir-6315, hsa-mir-4661, hsa-mir-1270, hsa-mir-710, hsa-mir-155, hsa-mir-16, hsa-mir-21, hsa-mir-223, hsa-mir-106a, hsa-mir-362, hsa-mir-320, hsa-mir-505, hsa-mir-132, hsa-mir-212, hsa-mir-105, hsa-mir-132, hsa-mir-135a, hsa-mir-142, hsa-mir-147, hsa-mir-155, hsa-mir-223, hsa-mir-298, hsa-mir-299a, hsa-mir-325, hsa-mir-410, hsa-mir-181b-1, hsa-mir-21, hsa-mir-219, hsa-mir-181a, hsa-mir-181b, hsa-mir-21, hsa-mir-132, hsa-mir-221, hsa-mir-146b, hsa-mir-221, hsa-mir-98, hsa-mir-9, hsa-mir-223, hsa-mir-181a, hsa-mir-203, hsa-mir-146b, hsa-mir-132, hsa-mir-132, hsa-mir-221, hsa-mir-27b, hsa-mir-146a, hsa-mir-210, hsa-mir-146a, hsa-mir-125b, hsa-mir-221, hsa-mir-579, hsa-mir-155, hsa-mir-24-1, hsa-mir-24-2, hsa-mir-33a, hsa-mir-33b, hsa-mir-1, hsa-mir-16, hsa-mir-206, hsa-mir-210, hsa-mir-132, hsa-mir-212, hsa-mir-146a, hsa-mir-147, hsa-mir-126, hsa-mir-125b, hsa-mir-219-2, hsa-mir-181a-2, hsa-mir-21, hsa-mir-155, hsa-mir-21, hsa-mir-146a, hsa-mir-146, hsa-mir-21, hsa-mir-21, hsa-mir-93, hsa-mir-155, hsa-mir-146a, hsa-mir-34a, hsa-mir-126, hsa-mir-33a, hsa-mir-148a, hsa-mir-31, hsa-mir-146a, hsa-mir-17, hsa-mir-18, hsa-mir-19a, hsa-mir-19b, hsa-mir-19b-1, hsa-mir-20a, hsa-mir-449c, hsa-mir-455, hsa-mir-92-1, hsa-mir-223, hsa-mir-29a, hsa-mir-34a, hsa-mir-424, hsa-mir-129, hsa-mir-186, hsa-mir-200c, hsa-mir-222, hsa-mir-101, hsa-mir-22, hsa-mir-100, hsa-mir-122, hsa-mir-143, hsa-mir-125b, hsa-mir-146a, hsa-mir-146a, hsa-mir-132, hsa-mir-221, hsa-mir-146a, hsa-mir-181, hsa-mir-21, hsa-mir-98, hsa-mir-155, hsa-mir-210, hsa-mir-146a, hsa-mir-223, hsa-mir-182, hsa-mir-183, hsa-mir-133a, hsa-mir-155, hsa-mir-210, hsa-mir-132, hsa-mir-608, hsa-let-7, hsa-mir-124, hsa-mir-146a, hsa-mir-155, hsa-mir-21, hsa-mir-223, hsa-mir-27b, hsa-mir-326, hsa-mir-155, hsa-mir-125b, hsa-mir-223, hsa-mir-181a, hsa-mir-146, hsa-mir-155, hsa-mir-34a, hsa-mir-132, hsa-mir-146b, hsa-mir-155, hsa-mir-21, hsa-mir-221, hsa-mir-223, hsa-mir-128, hsa-mir-320, hsa-mir-350, hsa-mir-34c, hsa-mir-223, hsa-mir-192, hsa-mir-105, hsa-mir-25, hsa-mir-17, hsa-mir-31, hsa-mir-135a, hsa-mir-181a, hsa-mir-181b, hsa-mir-199b, hsa-mir-204, hsa-mir-9, hsa-mir-146b, hsa-mir-219, hsa-mir-29a, hsa-mir-155, hsa-mir-221, hsa-mir-222, hsa-mir-150, hsa-let-7a-1, hsa-let-7a-2, hsa-let-7a-3, hsa-let-7b, hsa-let-7c, hsa-let-7d, hsa-let-7e, hsa-let-7f-1, hsa-let-7f-2, hsa-let-7g, hsa-let-7i, hsa-mir-21, hsa-mir-1236, hsa-mir-127, hsa-mir-181b, hsa-mir-23b, hsa-mir-203, hsa-mir-203, hsa-mir-224, hsa-mir-181a, hsa-mir-9, hsa-mir-146b, hsa-mir-182, hsa-mir-146, hsa-mir-149, hsa-mir-224, hsa-mir-708, hsa-mir-155, hsa-mir-142, hsa-mir-24, hsa-mir-30b, hsa-mir-125b, hsa-mir-148a, hsa-mir-125b, hsa-mir-132, hsa-mir-143, hsa-mir-148a, hsa-mir-193b, hsa-mir-223, hsa-mir-221, hsa-mir-146b, hsa-mir-221, hsa-mir-155, hsa-mir-193a, hsa-mir-194, hsa-mir-744, hsa-mir-130a, hsa-mir-142, hsa-mir-150, hsa-mir-320, hsa-mir-328, hsa-mir-16, hsa-mir-125a, hsa-mir-124, hsa-mir-200b, hsa-mir-125b, hsa-mir-16, hsa-mir-191a, hsa-mir-142, hsa-mir-223, hsa-mir-146a, hsa-mir-203, hsa-mir-146a, hsa-mir-26a, hsa-mir-132, hsa-mir-29b, hsa-mir-15a, hsa-mir-155, hsa-mir-155, hsa-mir-143, hsa-mir-124, hsa-mir-133, hsa-mir-1246, hsa-mir-595, hsa-mir-146a, hsa-mir-142, hsa-mir-122, hsa-mir-21, hsa-mir-155, hsa-mir-146a, hsa-mir-1-1, hsa-mir-1-2, hsa-mir-133a, hsa-mir-133b, hsa-mir-206, hsa-mir-146a, hsa-mir-455, hsa-mir-155, hsa-mir-146a, hsa-mir-155, hsa-mir-155, hsa-mir-223, hsa-let-7i, hsa-mir-145, hsa-mir-346, hsa-mir-10a, hsa-mir-155, hsa-mir-125b, hsa-mir-200, hsa-let-7e, hsa-mir-338, hsa-mir-92b, hsa-mir-99a, hsa-mir-99b, hsa-mir-146a, hsa-mir-146b, hsa-mir-146, hsa-mir-15b, hsa-mir-181b, hsa-mir-31, hsa-mir-106a, hsa-mir-19b-1, hsa-mir-19b-2, hsa-mir-629, hsa-mir-146a, hsa-mir-206, hsa-mir-31, hsa-mir-424, hsa-mir-21, hsa-mir-132, hsa-mir-31, hsa-mir-99b, hsa-mir-223, hsa-mir-143, hsa-mir-150, hsa-mir-155, hsa-mir-17, hsa-mir-18, hsa-mir-181, hsa-mir-19a, hsa-mir-19b-1, hsa-mir-20a, hsa-mir-223, hsa-mir-92-1, hsa-mir-150, hsa-mir-584, hsa-mir-766, hsa-mir-132, hsa-mir-130b, hsa-mir-132, hsa-mir-21, hsa-mir-34a, hsa-mir-132, hsa-mir-221, hsa-mir-222, hsa-mir-146a, hsa-mir-132, hsa-mir-223, and hsa-let-7b. In some embodiments, the cellular state is inflammation and the factor is a lncRNA selected from CBR3-AS1, CBR3-AS1, GAS5, GAS5, and NRON. In some embodiments, the cellular state is inflammation and the factor is a circRNA selected from circHECW2, HIPK2, and hsa-circRNA-2149.

In some embodiments, the cellular state is general brain damage and the factor is selected from pro-BDNF, pro-GDNF, pro-NGF, BDNF-AS, GDNS-AS, TALNEC2, and HIX003209. In some embodiments, the cellular state is general brain damage and the factor is a protein selected from pro-BDNF, pro-GDNF, and pro-NGF. In some embodiments, the cellular state is general brain damage and the factor is a lncRNA selected from BDNF-AS, GDNS-AS, TALNEC2, and HIX003209.

In some embodiments, the cellular state is stroke and the factor is selected from PRKCH, F2, ALOX5AP, F5, ACE, MTHFR, IL4, SELP, hsa-mir-4429, hsa-mir-2166, hsa-mir-4531, hsa-mir-4742, hsa-mir-4756, hsa-mir-4765, hsa-mir-5187, hsa-mir-5584, hsa-mir-1255a, circDLGAP4, Snhgl, Gas5, Dancr, Lnc-OGD2439, Snhgl2, Snhgl5, CDKN2B-AS10, and CDKN2B-AS11. In some embodiments, the cellular state is stroke and the factor is a miR selected from hsa-mir-4429, hsa-mir-2166, hsa-mir-4531, hsa-mir-4742, hsa-mir-4756, hsa-mir-4765, hsa-mir-5187, hsa-mir-5584, and hsa-mir-1255a. In some embodiments, the cellular state is stroke and the factor is the circRNA circDLGAP4. In some embodiments, the cellular state is stroke and the factor is a lncRNA selected from Snhg1, Gas5, Dancr, Lnc-OGD2439, Snhgl2, Snhgl5, CDKN2B-AS10, and CDKN2B-AS11. In some embodiments, the cellular state is stroke and the factor is a protein or mRNA encoding a protein selected from PRKCH, F2, ALOX5AP, F5, ACE, MTHFR, IL4 and SELP.

In some embodiments, the cellular state is neuroinflammation and the factor is selected from PTPRC, CD24, HLA-DRB1, HLA-A, hsa-mir-3614, hsa-mir-548ac, hsa-mir-648a, miR-181, let-7, miR-155, miR-21, miR-326, miR-10, miR-370, miR-146a, miR-15b, miR-210, miR-223, circ_0005402, circ_0035560, GSDMB ecircRNA, hsa_circ_0000517, hsa_circ_0000518, hsa_circ_0000519, hsa_circ_0001400, hsa_circ_0005402, hsa_circ_0024892, hsa_circ_0035560, hsa_circ_0056731, circHECW2, HIPK2, AP002765.1, Gm12478, Gm14005, mouselincRNA0681, mouselincRNA1117, SNORA40, and RN7SKRNA. In some embodiments, the cellular state is neuroinflammation and the factor is a protein or mRNA encoding a protein selected from PTPRC, CD24, HLA-DRB1 and HLA-A. In some embodiments, the cellular state is neuroinflammation and the factor is selected from hsa-mir-3614, hsa-mir-548ac, hsa-mir-648a, miR-181, let-7, miR-155, miR-21, miR-326, miR-10, miR-370, miR-146a, miR-15b, miR-210, miR-223, circ_0005402, circ_0035560, GSDMB ecircRNA, hsa_circ_0000517, hsa_circ_0000518, hsa_circ_0000519, hsa_circ_0001400, hsa_circ_0005402, hsa_circ_0024892, hsa_circ_0035560, hsa_circ_0056731, circHECW2, HIPK2, AP002765.1, Gm12478, Gm14005, mouselincRNA0681, mouselincRNA1117, SNORA40, and RN7SKRNA. In some embodiments, the cellular state is neuroinflammation and the factor is a miR selected from hsa-mir-3614, hsa-mir-548ac, hsa-mir-648a, miR-181, let-7, miR-155, miR-21, miR-326, miR-10, miR-370, miR-146a, miR-15b, miR-210, and miR-223. In some embodiments, the cellular state is neuroinflammation and the factor is a circRNA selected from circ_0005402, circ_0035560, GSDMB ecircRNA, hsa_circ_0000517, hsa_circ_0000518, hsa_circ_0000519, hsa_circ_0001400, hsa_circ_0005402, hsa_circ_0024892, hsa_circ_0035560, hsa_circ_0056731, circHECW2, and HIPK2. In some embodiments, the cellular state is neuroinflammation and the factor is a lncRNA selected from AP002765.1, Gm12478, Gm14005, mouselincRNA0681, mouselincRNA1117, SNORA40, and RN7SKRNA.

In some embodiments, the disease is Multiple Sclerosis and the factor is a protein or mRNA encoding a protein selected from PTPRC, CD24, HLA-DRB1 and HLA-A.

In some embodiments, the cellular state is neurodegeneration and the factor is selected from PRNP and SIX3-AS1. In some embodiments, the cellular state is neurodegeneration and the factor is the lncRNA SIX3-AS 1. In some embodiments, the cellular state is neurodegeneration and the factor is the protein or mRNA encoding PRNP. In some embodiments, the cellular state is neurodegeneration and the factor is selected from PRNP, APP, SORL1, UNC5C, DBN1, MAPT, PSEN1, HTT and SIX3-AS1. In some embodiments, the cellular state is neurodegeneration and the factor is a protein or mRNA encoding a protein selected from PRNP, APP, SORL1, UNC5C, DBN1, MAPT, HTT, and PSEN1.

In some embodiments, the disease is Cerebral amyloid angiopathy and the factor is a protein or mRNA encoding APP.

In some embodiments, the disease is Huntington’s Disease and the factor is a protein or mRNA encoding HTT.

In some embodiments, the disease is Alzheimer’s Disease and the factor is a protein or mRNA encoding a protein selected from SORL1, UNC5C, APP, and DBN1.

In some embodiments, the disease is Frontotemporal dementia and the factor is a protein or mRNA encoding a protein selected from MAPT, and PSEN1.

In some embodiments, the disease is major depressive disorder and the factor is selected from TPH2, CHRM2 NPAS2, AL442128.2, LINC00998, LINC01108, LINC02152, LINC02153, NONHSAG045500, NONHSAT142707, XLOC_009565, hsa_circRNA_002143, hsa_circRNA_100679, hsa_circRNA_102802, hsa_circRNA_103636, and hsa _circRNA_104953. In some embodiments, the disease is major depressive disorder and the factor is a lncRNA selected from AL442128.2, LINC00998, LINC01108, LINC02152, LINC02153, NONHSAG045500, NONHSAT142707, and XLOC_009565. In some embodiments, the disease is major depressive disorder and the factor is a circRNA selected from hsa_circRNA_002143, hsa_circRNA_100679, hsa_circRNA_102802, hsa_circRNA_103636, and hsa_circRNA_104953. In some embodiments, the disease is major depressive disorder and the factor is a protein or mRNA encoding a protein selected from TPH2, CHRM2 and NPAS2.

In some embodiments, the disease is coronary artery disease and the factor is CDKN2B-AS1. In some embodiments, the disease is coronary artery disease and the factor is the circRNA CDKN2B-AS1.

In some embodiments, the disease is cardiomyopathy and the factor is selected from HSPB6, LMNA, hsa-mir-3098, hsa-mir-467d, hsa-mir-1295a, ATP6V1G2-DDX39B, RP11-544D21.2, XLOC_014288, uc004cov.4, uc022bqu.1, and Titin circRNAs. In some embodiments, the disease is cardiomyopathy and the factor is a miR selected from hsa-mir-3098, hsa-mir-467d, and hsa-mir-1295a. In some embodiments, the disease is cardiomyopathy and the factor is a lncRNA selected from ATP6V1G2-DDX39B, RP11-544D21.2, XLOC_014288, uc004cov.4, and uc022bqu. 1. In some embodiments, the disease is cardiomyopathy and the factor is a Titin circRNA. In some embodiments, the disease is cardiomyopathy and the factor is a protein or mRNA encoding a protein selected from HSPB6 and LMNA.

In some embodiments, the disease is Heart disease and the factor is selected from CACNA1C, CREB1, DNM1L, DSP, GATA4, GJA1, HDAC2, HDAC5, JPH2, KCNH2, KRT18, MAP4, MAPK1, MYBPC3, MYL2, NOX4, PAFAH1B3, PON1, PRKAA1, PTPN11, RYR2, TNNI3, and TNNT2. In some embodiments, the disease is Heart disease and the factor is a protein or mRNA encoding a protein selected from CACNA1C, CREB1, DNM1L, DSP, GATA4, GJA1, HDAC2, HDAC5, JPH2, KCNH2, KRT18, MAP4, MAPK1, MYBPC3, MYL2, NOX4, PAFAH1B3, PON1, PRKAA1, PTPN11, RYR2, TNNI3, and TNNT2.

In some embodiments, the cellular state is renal fibrosis and the factor is selected from CAV1 and Snhg6. In some embodiments, the cellular state is renal fibrosis and the factor is the lncRNA Snhg6. In some embodiments, the cellular state is renal fibrosis and the factor is the protein or mRNA encoding CAV1.

In some embodiments, the disease is kidney disease and the factor is selected from PKD1, RASAL1, MET, SMAD2, KIRREL1, SMAD3, NPHS1, hsa-mir-687, hsa-mir-709 and MGAT3-AS1. In some embodiments, the disease is kidney disease and the factor is a miR selected from hsa-mir-687, and hsa-mir-709. In some embodiments, the disease is kidney disease and the factor is the lncRNA MGAT3-AS1. In some embodiments, the disease is kidney disease and the factor is a protein or mRNA encoding a protein selected from PKD1, RASAL1, MET, SMAD2, KIRREL1, SMAD3, and NPHS1.

In some embodiments, the disease is fatty liver disease and the factor is selected from CEBPA, SIRT1, LIVAR, RP11-484N16.1, uc.372, hsa-mir-209. In some embodiments, the disease is fatty liver disease and the factor is the miR hsa-mir-209. In some embodiments, the disease is fatty liver disease and the factor is a lncRNA selected from LIVAR, RP11-484N16.1, and uc.372. In some embodiments, the disease is fatty liver disease and the factor is a protein or mRNA encoding a protein selected from CEBPA and SIRT1.

In some embodiments, the cellular state is liver fibrosis and the factor is selected from hsa_circ_0013255, hsa_circ_0054345, hsa_circ_0061893, hsa_circ_0070963, hsa_circ_0071410, hsa_circ_0072765, and PALLD. In some embodiments, the cellular state is liver fibrosis and the factor is a circRNA selected from hsa_circ_0013255, hsa_circ_0054345, hsa_circ_0061893, hsa_circ_0070963, hsa_circ_0071410, hsa_circ_0072765, and PALLD.

In some embodiments, the cellular state is lung fibrosis and the factor is selected from CFTR and BGas. In some embodiments, the cellular state is lung fibrosis and the factor is the lncRNA BGas. In some embodiments, the cellular state is lung fibrosis and the factor is the protein or mRNA encoding CFTR. In some embodiments, the lung fibrosis is the disease cystic fibrosis. In some embodiments, cystic fibrosis is characterized by lung fibrosis. In some embodiments, the disease is cystic fibrosis and the factor is selected from CFTR and BGas. In some embodiments, the disease is cystic fibrosis and the factor is the lncRNA BGas. In some embodiments, the disease is cystic fibrosis and the factor is the protein or mRNA encoding CFTR.

In some embodiments, the method is for determining any one of (i) responsiveness of the subject to a medicament, (ii) predicting suitability of the EV to localize in a tissue; and (iii) cellular state of the cell of origin of said EV. In some embodiments, the method is for determining and/or predicting the progression of a disease by analyzing the response of different cells in culture or an animal model to these EVs. In some embodiments, the determining step may be performed in-vivo and/or in-vivo by assays known in the art.

In some embodiments, the cellular state is indicative of a pathological condition in the subject. In some embodiments, the pathological condition is a disease. In some embodiments, the disease is a disease provided in Table 2. In some embodiments, the pathological condition is a microbial infection and the method is for determining the cells and tissues infected by the microbe. In some embodiments, the subject suffers from a disease and the cellular state is indicative of disease activity. In some embodiments, the cellular state is indicative of disease activity in the tissue, organ or cell type. In some embodiments, the subject suffers from a disease and the cellular state is indicative of disease symptoms. In some embodiments, disease symptoms are disease symptoms that are to develop. In some embodiments, disease symptoms are imminent disease symptoms. In some embodiments, the cellular state is indicative of disease symptoms in the tissue, organ or cell-type.

In some embodiments, the method is a method of predicting disease spread. In some embodiments, disease spread is metastasis. In some embodiments, disease spread is symptomatic disease spread. In some embodiments, the method is a method of predicting the development of symptoms. In some embodiments, method is a method of early disease detection. In some embodiments, the method is a method of early symptom detection. In some embodiments, symptom detection is detection of onset of symptoms. In some embodiments, the method is a method of detection of residual disease. In some embodiments, the method is a method of detecting disease relapse. In some embodiments, disease relapse is cancer relapse. In some embodiments, early detection is detection before development of symptoms. In some embodiments, early detection is detection before conventional methods can detect.

In some embodiments, the subject suffers from a disease and is receiving treatment for the disease. In some embodiments, the method is a method of determining effectiveness of the treatment. In some embodiments, the method is a method of determining responsiveness of the subject to the treatment. In some embodiments, the method comprises performing the method of the invention at a first time point and a second time point. In some embodiments, the method comprises comparing the cellular states at the two time points. In some embodiments, the method comprises comparing the at least one factor at the two time points. In some embodiments, comparing the cellular state comprises comparing the at least one factor. In some embodiments, comparing the at least one factor is comparing the abundance of the factor. In some embodiments, comparing the at least one factor is comparing the concentration of the factor. In some embodiments, the first time point is before commencing treatment. In some embodiments, the first time point is early in treatment. In some embodiments, the second time point is after treatment has commenced. In some embodiments, the second time point is later in treatment. In some embodiments, the second time point is later than the first time point. In some embodiments, the second time point is after more treatment than the first time point. In some embodiments, a decrease in the cellular state in the subject indicates the effectiveness of the treatment. In some embodiments, a decrease in the cellular state in the subject indicates responsiveness to the treatment. In some embodiments, a decrease is from the first time point to the second time point. In some embodiments, an increase at the second time point or a lack of decrease indicates the treatment is not effective or lack of responsiveness to the treatment.

In some embodiments, the method is a method of detecting a side effect of a therapeutic agent. In some embodiments, the method is a method of detecting the efficacy of a therapeutic agent. In some embodiments, the subject is receiving a therapeutic agent and detecting a cellular state in a tissue, organ or cell-type that is not the target of the therapeutic agent is indicative of a side effect. In some embodiments, the side effect is an off-target effect. In some embodiments, the subject is receiving a therapeutic agent and detecting a cellular state in a tissue, organ or cell-type that is the target of the therapeutic agent is indicative of the efficacy of the therapeutic agent. In some embodiments, a decrease in the cellular state in a target tissue, organ or cell-type is indicative that the therapeutic agent is effective. In some embodiments, the therapeutic agent is a vaccine. In some embodiments, the therapeutic agent is an anticancer agent. In some embodiments, the therapeutic agent is a small molecule therapeutic agent. In some embodiments, the therapeutic agent is a biologic.

In some embodiments, the method further comprises treating said subject with a suitable treatment based on any one of the following determinations: pathological condition, cellular state, tissue, organ, or cell of origin, or a combination thereof. In some embodiments, the method further comprises administering to the subject a treatment. In some embodiments, the treatment is selected to treat the cellular condition. In some embodiments, the treatment is selected to treat the tissue, organ or cell type. In some embodiments, the treatment is selected to treat the cellular condition in the tissue, organ or cell type. In some embodiments, the treatment is selected to treat the disease. In some embodiments, the treatment is a therapeutic agent. In some embodiments, the treatment comprises administering to the subject a pharmaceutical composition capable of treating the cellular condition or disease. In some embodiments, the treatment is a therapeutic EV. In some embodiments, the treatment is a pharmaceutical composition comprising a therapeutic EV. In some embodiments, a therapeutic EV is an EV derived from a mesenchymal stromal cell (MSC). MSCs are also known as mesenchymal stem cells. In some embodiments, the MSC is a chorionic placenta MSC. In some embodiments, the MSC is an umbilical cord MSC. In some embodiments, the MSC is selected from a chorionic placenta, umbilical cord, bone marrow, dental pulp, adipose, and amniotic placenta. In some embodiments, the EV is derived from milk. In some embodiments, the milk is bovine milk. In some embodiments, the milk is human milk. In some embodiments, the EV is derived from a plant.

As used herein, the term “mesenchymal stromal cell”, “mesenchymal stem cell” or “MSC”, refers to multipotent stromal stem cells that have the ability to differentiate into osteoblasts, adipocytes, myocytes, chondroblasts, skeletal muscle cells and endothelial cells. MSC are present in the bone marrow, adipose tissue, peripheral blood, chorionic placenta (CH), amniotic placenta, umbilical cord (UC) blood, and dental pulp, among other tissues. The term “multipotent” refers to stem cells which are capable of giving rise to many cell types. In some embodiments, the MSC is derived from umbilical cord or chorionic placenta. In some embodiments, the MSC is derived from dental pulp, umbilical cord or chorionic placenta. In some embodiments, the MSC is derived from chorionic placenta. In some embodiments, the MSC is derived from umbilical cord. In some embodiments, the MSC is derived from dental pulp. In some embodiments, the MSC is derived from any one of umbilical cord and chorionic placenta. In some embodiments, the MSC is not derived from amniotic placenta. In some embodiments, MSC is derived from placenta. In some embodiments, the MSC is derived from amniotic placenta. In some embodiments, the MSC is derived from bone marrow. In some embodiments, the MSC is derived from adipose tissue. In some embodiments, the MSC is derived from umbilical cord blood. In some embodiments, the MSC is derived from peripheral blood. In some embodiments, the pharmaceutical composition is devoid of amniotic placenta MSCs. In some embodiments, the pharmaceutical composition is substantially devoid of amniotic placenta MSCs. In some embodiments, the MSC is not an adipose derived MSC. In some embodiments, the MSC is an unmodified MSC. In some embodiments, the MSC has been modified to over-express at least one exogenous molecule.

In some embodiments, the MSC is derived from a stem cell. In some embodiments, the MSC is differentiated from a stem cell. In some embodiments, the stem cell is a naturally occurring stem cell. In some embodiments, the stem cell is a human stem cell. In some embodiments, the stem cell is an adult stem cell. In some embodiments, the stem cell is an embryonic stem cell. In some embodiments, the stem cell is not an embryonic stem cell. In some embodiments, the stem cell is an umbilical cord stem cell. In some embodiments, the stem cell is a placental stem cell. In some embodiments, the stem cell is an induced pluripotent stem cell (iPSC). In some embodiments, the stem cell is a non-naturally occurring stem cell. In some embodiments, the MSC is derived from an iPSC. In some embodiments, MSC is differentiated from an iPSC.

In some embodiments, the MSC and/or its extracellular vesicles are allogenic to the subject. In some embodiments, the MSC and/or its EVs are autologous to the subject. In some embodiments, the MSC and/or its EVs are allogenic or autologous to the subject. In some embodiments, the MSC and/or its EVs do not induce an immune response in the subject. MSC and especially their extracellular vesicles have a strong advantage as a therapeutic as they do not express MHCII molecules and do not induce an immune response. Further MSCs and their EVs actively inhibit the immune response. CH and UC MSCs and their EVs are particularly effective in this respect. In this way the MSCs and/or their exosomes can be used as an “off the shelf” therapeutic agent that can be administered to any subject in need thereof.

Chorionic (CH), and umbilical cord (UC) MSCs are well known in the art. In some embodiments, these MSCs or their secreted vesicles can be identified by examining the expression of various proteins, and regulatory RNA such as are described in international patent application WO/2018083700, the content of which are herein incorporated by reference in their entirety. In some embodiments, the MSCs are identified by the tissue they were isolated from.

In some embodiments, the MSC is an umbilical cord (UC) MSC. In some embodiments, the MSC is a chorionic placenta (CH) MSC.

In some embodiments, the method is for determining and/or predicting the progression of a disease by analyzing the response of different cells in culture or an animal model to these EVs.

In some embodiments, the pathological condition is a microbial infection, including but not limited to bacteria, virus, parasites, and prions. In some embodiments, the method the microbial infection is a viral infection. In some embodiments, the method the viral infection is SARS-CoV-1, or SARS-CoV-2. In some embodiments, the microbial infection is a viral infection and wherein the at least one parameter is selected from viral DNA, mRNAs, viral microRNAs (vmiRNA), nonprotein coding RNAs (vRNA), full-length genomic RNA (gRNA), and virus-specific proteins. In some embodiments, the microbial infection is a viral infection and wherein the at least one parameter is selected from viral DNA, mRNAs, viral microRNAs (vmiRNA), nonprotein coding RNAs (vRNA), full-length genomic RNA (gRNA), and virus-specific proteins, associated with SARS-CoV-1, or SARS-CoV-2.

In some embodiments, the isolation of EVs with specific viral proteins and tissue, organ, or cell-originating marker (e.g., such as determining the origin of the EV as being lung, kidney or CNS origin) enables the monitoring of viral spreading and infection in these specific tissues.

In some embodiments, a pharmaceutical composition comprises a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer’s solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer’s solution, dextrose solution, Hank’s solution, and DMSO. These additional inactive components, as well as effective formulations and administration procedures, are well known in the art and are described in standard textbooks, such as Goodman and Gillman’s: The Pharmacological Bases of Therapeutics, 8th Ed., Gilman et al. Eds. Pergamon Press (1990); Remington’s Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the inflammation is a result of an infection such as a virus including but not limited to SARS-CoV-1 or SARS-CoV-2.

In some embodiments, the fibrosis (e.g., lung fibrosis) is a result of ARDS. In some embodiments, the fibrosis (e.g., lung, cardiac, kidney fibrosis) is a result of cytokine storm. In some embodiments, the fibrosis (e.g., lung, cardiac, kidney fibrosis) is a result of a severe hyperinflammatory responses (e.g., to a virus including but not limited to SARS-CoV-1 or SARS-CoV-2). In some embodiments, the fibrosis (e.g., lung, cardiac, kidney fibrosis) is a result of a chronic pathological condition.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject’s quality of life.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with a fractured bone, a bone injury, diminished bone mass and/or bone abnormality.

As used herein, the terms “subject” or “individual” or “animal” or “patient” or “mammal,” refers to any subject, particularly a mammalian subject, for whom therapy is desired, for example, a human.

In some embodiments, a subject in need thereof is infected with a viral infection. In some embodiments, a subject in need thereof is infected with a coronavirus. In some embodiments, a subject in need thereof is infected with a coronavirus caused by SARS-CoV-1. In some embodiments, a subject in need thereof is infected with a coronavirus caused by SARS-CoV-2. In some embodiments, the viral infection is an infection by a virus bound by the virus binding protein.

The term “administering” as used herein refers to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, inhaled, intramuscular, anal, oral, intrathecal intranasal, or intraperitoneal. In some embodiments, the administration is intravenous. In some embodiments, the administration is intranasal. In some embodiments, the administration is intrathecal. In some embodiments, the administration is intramuscular. In some embodiments, the administration is oral. In some embodiments, the administration is by inhalation. In some embodiments, the administering is systemic administration or administration to a site of infection. In some embodiments, administration is systemic administration. In some embodiments, administration is administration to a site of infection. In some embodiments, administration is to the lungs.

The dosage administered will be dependent upon the age, health, and weight of the recipient, kind of concurrent treatment, if any, frequency of treatment, and the nature of the effect desired.

As used herein, the term “about” when combined with a value refers to plus and minus 10% of the reference value. For example, a length of about 1000 nanometers (nm) refers to a length of 1000 nm+- 100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

### Example 1: Use of EVs for Diagnosis and Personalized Treatment

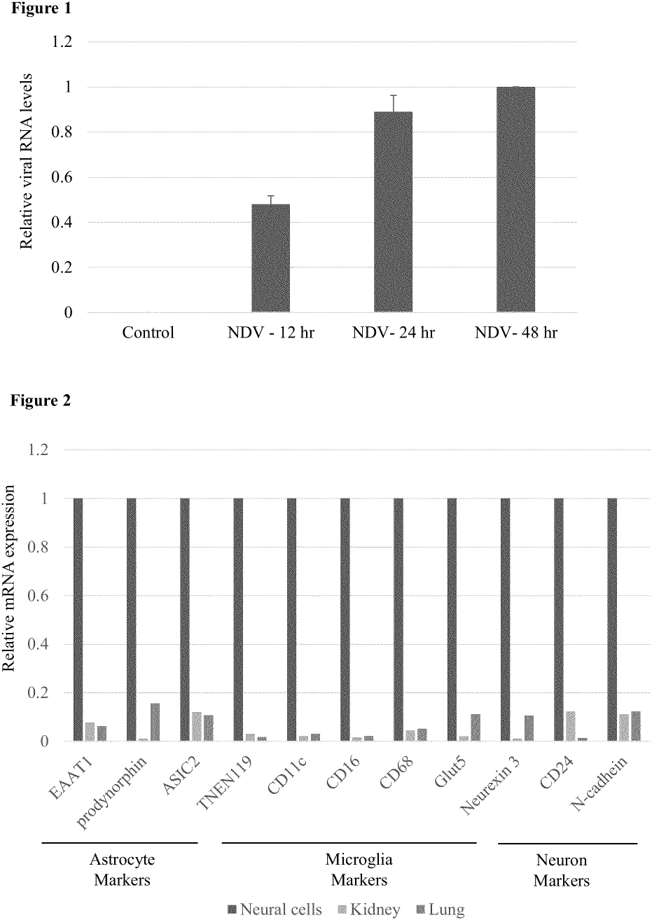

In order to test the utility of extracellular vesicles (EVs) as a source of information on viral spread and damage, the monocyte cell line THP-1 was infected with Newcastle Disease Virus (NDV) and EVs were collected at various time points. Uninfected THP-1 cells were used as a negative control. The collected EVs were lysed, and PCR was performed using primers specific for NDV. NDV did not induce cell death in THP-1 cells within the time frames examined in this experiment, and so analysis of cfDNA would not be informative in assessing infection. However, as can clearly be seen in FIG. 1, THP-1 cells infected with NDV secrete EVs containing NDV RNA. Further, the level of RNA within the EVs was indicative of the extent of infection. This means that EVs can be used diagnostically to detect infectious agents in a subject.

It would be even more informative to be able to match the EVs containing an infectious agent to their tissue/cell type of origin. To this end, an in-silico analysis of markers that can uniquely identify the tissue/cell-type of origin of EVs was carried out. Various databases including the Human Protein Atlas, the Human microRNA Disease Database (HMDD), and the LncRNADisease Database were mined for information on cell/tissue/organ expression, localization and specificity using a machine learning algorithm. The association with disease and various adverse conditions was also probed. A resulting list of markers and factors was cross-referenced with supporting experimental data, in particular in the case of lncRNAs and circRNAs for which predictive algorithms notoriously produce false positives. Further, direct omics analysis was performed on various tissues and cell-types in order to corroborate identified markers and factors. These validation studies produced the lists of markers and factors provided hereinbelow. The use of experimental validation was essential, as surface markers were found that although uniquely expressed on a certain cell-type were not expressed on EVs from that cell type. For example, TSPO is marker for microglial cells, however, when examined it was found that microglia derived EVs do not express TSPO.

This analysis resulted in the identification of a large number of surface markers that can be used to identify the origin of EVs found in a biological sample. These surface markers are summarized in Tables 1.

TABLE 1

Unique surface markers for identification of EVs from tissues/cell-types.

| | |
|---|---|
| Surface Protein Markers | Tissue/cell-type |
| ADAM11, AKAP5, APLP1, ASIC2, ATP2B3, BCAN, CACNG8, CASKIN1, CNTN2, CNTNAP2, DLG4, DRD2, DRD5, ERC2, GABRA1, GABRA3, GABRB2, GPR158, GPR26, GRIA2, GRIK4, GRM2, GRM4, HCRTR2, HS3ST5, HTR6, KCNA1, KCNA2, KCNA3, KCNF1, KCNG2, KCNJ3, KCNJ9, KCNK13, LEPR, MC3R, MLC1, MYO15A, NCAN, NPTXR, NRSN1, NRXN3, PACSIN1, PDYN, PIEZO1, PLXNB3, PRL, PTPN5, PTPRZ1, RGS7, SCN2A, SLC12A5, SLC1A2, SLC1A3, SLC6A11, SLC6A17, SLC8A2, SLITRK1, SNRPN, SNTG1, SYT1, TAGLN3, TMEM59L, TRHR | Brain |
| PDYN, SNRPN, TRHR, ASIC2, SNTG1, GRM2, PTPN5, NRXN3, PTPRZ1, SLC1A2, RGS7, AQP4, SLC1A2 | Astrocytes |
| TMEM119, CR3, CSF1R, Siglec-H, CX3CR1, CD200, P2yR12, Tyro3, AXL, Mertk, Cd11c, CD68, CD11b, CD80, CD115, CD16, FCER1G, CD16, CA115, EMR1, TREM2, RCA1, Glut5, CD32 | Microglia |
| Kv2.2, Kv2.1, Kv4.3, Nav1.4, SLC1A3, N-Cadherin, NG-2, synaptophysin, PSD95, vGluT1, vGlut2, NMDR1, NMDAR2B, GAT1, GABAb receptor 1, GAGAb receptor 2, DAT, CD15, CD24, CD29 | Neurons |
| MYL4, NPPA | Heart |
| AQP6, ATP6V1G3, CLDN16, FGB, NPHS1, NPHS2, SLC22A12, SLC22A13, SLC22A2, SLC22A6, SLC22A8, SLC34A1, SLC34A3, SLC4A1, UMOD | Kidney |
| ABCB4, ASGR2, C4B, CPB2, F2, HP, HPR, IGFALS, LPA, MAT1A, MBL2, PROZ, SLC10A1, SLC13A5, SLC27A5, SLCO1B1, SLCO1B3, UROC1 | Liver |
| ACP5, SCGB1A1, SFTTPB | Lung |

The analysis also resulted in the identification of a large number of factors that uniquely identify particular cellular states either in general or in particular tissues. Many of the proteins undergo post-translational modification and these modifications can be disease specific. Many disease markers were found across a variety of disease/conditions and as such factors were selected that appeared in less than 4 pathologies/conditions. These factors are summarized in Table 2. As results specific for some tissues/diseases were limited, and in such cases the combined use of multiple less specific molecule can also be employed.

TABLE 2

Factors that identify particular cellular states.

| | | |
|---|---|---|
| Brain | Factors | Type |
| -General | pro-BDNF, pro-GDNF, pro-NGF | Proteins |
| | BDNF-AS, GDNS-AS, TALNEC2, HIX003209 | lncRNAs |
| -Stroke | hsa-mir-4429, hsa-mir-2166, hsa-mir-4531, hsa-mir-4742, hsa-mir-4756, hsa-mir-4765, hsa-mir-5187, hsa-mir-5584, hsa-mir-1255a | miRs |
| | circDLGAP4 | circRNAs |
| | Snhgl, Gas5, Dancr, Lnc-OGD2439, Snhgl2, Snhgl5, CDKN2B-AS 10, CDKN2B-AS 11 | lncRNAs |
| | PRKCH, F2, ALOX5AP, F5, ACE, MTHFR, IL4, SELP | Proteins |
| -Neuroinflammation | hsa-mir-3614, hsa-mir-548ac, hsa-mir-648a, miR-181, let-7, miR-155, miR-21, miR-326, miR-10, miR-370, miR-146a, miR-15b, miR-210, miR-223 | miRs |
| | circ_0005402, circ_0035560, GSDMB ecircRNA, hsa_circ_0000517, hsa_circ_0000518, hsa_circ_0000519, hsa_circ_0001400, hsa _circ_0005402, hsa _circ_0024892, hsa_circ_0035560, hsa_circ_0056731, circHECW2, HIPK2 | circRNAs |
| | AP002765.1, Gm12478, Gm14005, mouselincRNA0681, mouselincRNA1117, SNORA40, RN7SKRNA | lncRNAs |
| -Neuroinflammation/ Multiple Sclerosis | PTPRC, CD24, HLA-DRB 1 HLA-A | Proteins |
| -Neurodegeneration | SIX3-AS1 | IncRNAs |
| | PRNP | Protein |
| -Neurodegeneration/ Cerebral amyloid angiopathy | APP | Protein |
| -Neurodegeneration/ Alzheimer’s Disease | SORL1, UNC5C, APP, DBN1 | Proteins |
| -Neurodegeneration/ Frontotemporal dementia | MAPT, PSEN1 | Proteins |
| -Neurodegeneration/ Huntington’s Disease | HTT | Proteins |
| -Major depressive disorder | AL442128.2, LINC00998, LINC01108, LINC02152, LINC02153, NONHSAG045500, NONHSAT142707, XLOC_009565 | IncRNAs |
| | hsa_circRNA_002143, hsa _circRNA_100679, hsa_circRNA_102802, hsa _circRNA_103636, hsa_circRNA_104953 | circRNAs |
| | TPH2, CHRM2 NPAS2 | Proteins |
| -Coronary artery disease | CDKN2B-AS1 | circRNA |
| -Cardiomyopathy | hsa-mir-3098, hsa-mir-467d, hsa-mir-1295a | miRs |
| | ATP6V1G2-DDX39B, RP11-544D21.2, XLOC_014288, uc004cov.4, uc022bqu.1 | IncRNAs |
| | Titin circRNAs | circRNAs |
| | HSPB6, LMNA | Proteins |
| -Heart disease | CACNA1C, CREB1, DNM1L, DSP, GATA4, GJA1, HDAC2, HDAC5, JPH2, KCNH2, KRT18, MAP4, MAPK1, MYBPC3, MYL2, NOX4, PAFAH1B3, PON1, PRKAA1, PTPN11, RYR2, TNNI3, TNNT2 | Proteins |
| -Renal fibrosis | Snhg6 | lncRNA |
| | CAV1 | Protein |
| -Kidney disease | hsa-mir-687, hsa-mir-709 | miR |
| | MGAT3-AS1 | lncRNA |
| | PKD1, RASAL1, MET, SMAD2, KIRREL1, SMAD3, NPHS 1 | Proteins |
| -Liver fibrosis | hsa_circ_0013255, hsa_circ_0054345, hsa_circ_0061893, hsa_circ_0070963, hsa_circ_0071410, hsa_circ_0072765, PALLD | circRNAs |
| -Fatty liver disease | hsa-mir-209 | miRs |
| | LIVAR, RP11-484N16.1, uc.372 | IncRNAs |
| | CEBPA, SIRT1 | Proteins |
| -Cystic fibrosis | BGas | IncRNAs |
| | CFTR | Protein |
| | hsa-mir-3560, hsa-mir-3588, hsa-mir-466b, hsa-mir-6315, hsa-mir-4661, hsa-mir-1270, hsa-mir-710, hsa-mir-155, hsa-mir-16, hsa-mir-21, hsa-mir-223, hsa-mir-106a, hsa-mir-362, hsa-mir-320, hsa-mir-505, hsa-mir-132, hsa-mir-212, hsa-mir-105, hsa-mir-132, hsa-mir-135a, hsa-mir-142, hsa-mir-147, hsa-mir-155, hsa-mir-223, hsa-mir-298, hsa-mir-299a, hsa-mir-325, hsa-mir-410, hsa-mir-181b-1, hsa-mir-21, hsa-mir-219, hsa-mir-181a, hsa-mir-181b, hsa-mir-21, hsa-mir-132, hsa-mir-221, hsa-mir-146b, hsa-mir-221, hsa-mir-98, hsa-mir-9, hsa-mir-223, hsa-mir-181a, hsa-mir-203, hsa-mir-146b, hsa-mir-132, hsa-mir-132, hsa-mir-221, hsa-mir-27b, hsa-mir-146a, hsa-mir-210, hsa-mir-146a, hsa-mir-125b, hsa-mir-221, hsa-mir-579, hsa-mir-155, hsa-mir-24-1, hsa-mir-24-2, hsa-mir-33a, hsa-mir-33b, hsa-mir-1, hsa-mir-16, hsa-mir-206, hsa-mir-210, hsa-mir-132, hsa-mir-212, hsa-mir-146a, hsa-mir-147, hsa-mir-126, hsa-mir-125b, hsa-mir-219-2, hsa-mir-181a-2, hsa-mir-21, hsa-mir-155, hsa-mir-21, hsa-mir-146a, hsa-mir-146, hsa-mir-21, hsa-mir-21, hsa-mir-93, hsa-mir-155, hsa-mir-146a, hsa-mir-34a, hsa-mir-126, hsa-mir-33a, hsa-mir-148a, hsa-mir-31, hsa-mir-146a, hsa-mir-17, hsa-mir-18, hsa-mir-19a, hsa-mir-19b, hsa-mir-19b-1, hsa-mir-20a, hsa-mir-449c, hsa-mir-455, hsa-mir-92-1, hsa-mir-223, hsa-mir-29a, hsa-mir-34a, hsa-mir-424, hsa-mir-129, hsa-mir-186, hsa-mir-200c, hsa-mir-222, hsa-mir-101, hsa-mir-22, hsa-mir-100, hsa-mir-122, hsa-mir-143, hsa-mir-125b, hsa-mir-146a, hsa-mir-146a, hsa-mir-132, hsa-mir-221, hsa-mir-146a, hsa-mir-181, hsa-mir-21, hsa-mir-98, hsa-mir-155, hsa-mir-210, hsa-mir-146a, hsa-mir-223, hsa-mir-182, hsa-mir-183, hsa-mir-133a, hsa-mir-155, hsa-mir-210, hsa-mir-132, hsa-mir-608, hsa-let-7, hsa-mir-124, hsa-mir-146a, hsa-mir-155, hsa-mir-21, hsa-mir-223, hsa-mir-27b, hsa-mir-326, hsa-mir-155, hsa-mir-125b, hsa-mir-223, hsa-mir-181a, hsa-mir-146, hsa-mir-155, hsa-mir-34a, hsa-mir-132, hsa-mir-146b, hsa-mir-155, hsa-mir-21, hsa-mir-221, hsa-mir-223, hsa-mir-128, hsa-mir-320, hsa-mir-350, hsa-mir-34c, hsa-mir-223, hsa-mir-192, hsa-mir-105, hsa-mir-25, hsa-mir-17, hsa-mir-31, hsa-mir-135a, hsa-mir-181a, hsa-mir-181b, hsa-mir- | miRs |
| | 199b, hsa-mir-204, hsa-mir-9, hsa-mir-146b, hsa-mir-219, hsa-mir-29a, hsa-mir-155, hsa-mir-221, hsa-mir-222, hsa-mir-150, hsa-let-7a-1, hsa-let-7a-2, hsa-let-7a-3, hsa-let-7b, hsa-let-7c, hsa-let-7d, hsa-let-7e, hsa-let-7f-1, hsa-let-7f-2, hsa-let-7g, hsa-let-7i, hsa-mir-21, hsa-mir-1236, hsa-mir-127, hsa-mir-181b, hsa-mir-23b, hsa-mir-203, hsa-mir-203, hsa-mir-224, hsa-mir-181a, hsa-mir-9, hsa-mir-146b, hsa-mir-182, hsa-mir-146, hsa-mir-149, hsa-mir-224, hsa-mir-708, hsa-mir-155, hsa-mir-142, hsa-mir-24, hsa-mir-30b, hsa-mir-125b, hsa-mir-148a, hsa-mir-125b, hsa-mir-132, hsa-mir-143, hsa-mir-148a, hsa-mir-193b, hsa-mir-223, hsa-mir-221, hsa-mir-146b, hsa-mir-221, hsa-mir-155, hsa-mir-193a, hsa-mir-194, hsa-mir-744, hsa-mir-130a, hsa-mir-142, hsa-mir-150, hsa-mir-320, hsa-mir-328, hsa-mir-16, hsa-mir-125a, hsa-mir-124, hsa-mir-200b, hsa-mir-125b, hsa-mir-16, hsa-mir-191a, hsa-mir-142, hsa-mir-223, hsa-mir-146a, hsa-mir-203, hsa-mir-146a, hsa-mir-26a, hsa-mir-132, hsa-mir-29b, hsa-mir-15a, hsa-mir-155, hsa-mir-155, hsa-mir-143, hsa-mir-124, hsa-mir-133, hsa-mir-1246, hsa-mir-595, hsa-mir-146a, hsa-mir-142, hsa-mir-122, hsa-mir-21, hsa-mir-155, hsa-mir-146a, hsa-mir-1-1, hsa-mir-1-2, hsa-mir-133a, hsa-mir-133b, hsa-mir-206, hsa-mir-146a, hsa-mir-455, hsa-mir-155, hsa-mir-146a, hsa-mir-155, hsa-mir-155, hsa-mir-223, hsa-let-7i, hsa-mir-145, hsa-mir-346, hsa-mir-10a, hsa-mir-155, hsa-mir-125b, hsa-mir-200, hsa-let-7e, hsa-mir-338, hsa-mir-92b, hsa-mir-99a, hsa-mir-99b, hsa-mir-146a, hsa-mir-146b, hsa-mir-146, hsa-mir-15b, hsa-mir-181b, hsa-mir-31, hsa-mir-106a, hsa-mir-19b-1, hsa-mir-19b-2, hsa-mir-629, hsa-mir-146a, hsa-mir-206, hsa-mir-31, hsa-mir-424, hsa-mir-21, hsa-mir-132, hsa-mir-31, hsa-mir-99b, hsa-mir-223, hsa-mir-143, hsa-mir-150, hsa-mir-155, hsa-mir-17, hsa-mir-18, hsa-mir-181, hsa-mir-19a, hsa-mir-19b-1, hsa-mir-20a, hsa-mir-223, hsa-mir-92-1, hsa-mir-150, hsa-mir-584, hsa-mir-766, hsa-mir-132, hsa-mir-130b, hsa-mir-132, hsa-mir-21, hsa-mir-34a, hsa-mir-132, hsa-mir-221, hsa-mir-222, hsa-mir-146a, hsa-mir-132, hsa-mir-223, hsa-let-7b | |
| | CBR3-AS1, CBR3-AS1, GAS5, GAS5, NRON | lncRNAs |
| | circHECW2, HIPK2, hsa-circRNA-2149 | circRNAs |
| | STAT3 | Protein |
| | hsa_circ_0013255, hsa_circ_0054345, hsa_circ_0061893, hsa_circ_0070963, hsa_circ_0071410, hsa_circ_0071410, hsa_circ_0072765, PALLD | circRNAs |
| | BGas, CDKN2B-AS1, H19, MEG3, uc.77, WT1-AS | lncRNAs |
| | CFTR | Protein |
| | hsa-mir-19a, hsa-mir-29, hsa-mir-18a, hsa-mir-155, hsa-mir-21, hsa-mir-214, hsa-mir-29b, hsa-mir-196a, hsa-mir-196b, hsa-mir-214, hsa-mir-19b, hsa-mir-19a, hsa-mir-19b, hsa-mir-26b, hsa-mir-29a, hsa-mir-145, hsa-mir-449a, hsa-mir-185, hsa-mir-186, hsa-mir-29a, hsa-mir-221, hsa-mir-208a, hsa-mir-25, hsa-mir-29a, hsa-mir-21, hsa-mir-23b, hsa-mir-27b, hsa-mir-30b, hsa-mir-21, hsa-mir-382, hsa-mir-155, hsa-mir-29a, hsa-mir-29b-1, hsa-mir-29b-2, hsa-mir-29c, hsa-mir-21, hsa-mir-21, hsa-mir-21, hsa-mir-31, hsa-mir-101-1, hsa-mir-101-2, hsa-mir-21, hsa-mir-33a, hsa-mir-214, hsa-mir-218, hsa-mir-509, hsa-mir-26a, hsa-mir-106b, hsa-mir-215, hsa-mir-199a, hsa-let-7e, hsa-mir-125a, hsa-mir-99b, hsa-mir-21, hsa-mir-29c, hsa-mir-153, hsa-mir-200, hsa-mir-486, hsa-mir-21, hsa-let-7d, hsa-mir-34a, hsa-mir-146a, hsa-mir-155, hsa-mir-370, hsa-mir-708, hsa-mir-378, hsa-mir-34a, hsa-mir-200c, hsa-mir-34a, hsa-let-7d, hsa-mir-154, hsa-mir-24, hsa-mir-18a, hsa-mir-29c, hsa-mir-181b, hsa-mir-23a, hsa-mir-27a, hsa-mir-199, hsa-mir-135a, hsa-mir-200b, hsa-mir-21, hsa-mir-410, hsa-mir-142, hsa-mir-222, hsa-mir-21, hsa-mir-200c, hsa-mir-196a, hsa-mir-30, hsa-mir-30e, hsa-mir-122, hsa-mir-326, hsa-mir-101, hsa-mir-92a, hsa-mir-29b, hsa-mir-203, hsa-mir-144, hsa-mir-193, hsa-mir-30, hsa-mir-130a, hsa-mir-27b, hsa-mir-1908, hsa-mir-21, hsa-mir-130b, hsa-mir-21, hsa-mir-134, hsa-mir-155, hsa-mir-9, hsa-mir-29c, hsa-mir-21, hsa-mir-18a, hsa-mir-98, hsa-mir-30a, hsa-mir-21, hsa-mir-29b, hsa-mir-30a, hsa-mir-152, hsa-mir-26a, hsa-mir-146a, hsa-mir-34a, hsa-mir-29b, hsa-mir-30d, hsa-mir-942, hsa-mir-7, hsa-mir-21, hsa-mir-200a, hsa-mir-542, hsa-mir-200, hsa-mir-454, hsa-mir-31, hsa-mir-133, hsa-mir-29, hsa-mir-21, hsa-mir-17, hsa-mir-16, hsa-mir-17, hsa-mir-27b, hsa-mir-21, hsa-mir-1343, hsa-mir-125b, hsa-mir-142, hsa-mir-21, hsa-mir-221, hsa-mir-223, hsa-mir-34a, hsa-mir-433, hsa-mir-130a, hsa-mir-29a, hsa-let-7d, hsa-mir-192, hsa-mir-26a, hsa-mir-210, hsa-mir-214, hsa-mir-140, hsa-mir- | miRs |
| | 21, hsa-mir-133a, hsa-mir-30a, hsa-mir-18a, hsa-mir-212, hsa-mir-203, hsa-mir-221, hsa-mir-222, hsa-mir-21, hsa-mir-149, hsa-mir-155, hsa-mir-34, hsa-mir-34, hsa-mir-34a | |

These markers and factors were confirmed in cells. First, primary human neuronal cells (a mix of astrocytes, microglia and mature neurons), primary kidney cells and primary human lung cells were cultured and EVs were isolated from the media. PCR was performed on the EVs to detect several cell-type specific markers, that is markers specific for astrocytes, microglia and mature neurons. These markers were only significantly detected in the EVs from the neuronal coculture (FIG. 2) indicating that tissue/cell type specific protein expression within EVs can be used to determine the EVs origin. By combining this with the presence of an infectious agent marker (DNA/RNA/protein) the main site of infection can be determined.

This diagnostic approach extends beyond detection of an infectious agent. Markers of cellular states, such as inflammation, hypoxia, hyper-proliferation and the like can also be detected in circulating EVs. Thus, EVs can be used to monitor tissue damage without the presence of cell death (and certainly with it), as well as disease progression and treatment efficacy.

### Example 2: Specific Uses of the Diagnostic Method

Blood or another biological fluid is received from a patient with a known condition/disease, e.g., a viral infection. Although the subject is currently asymptomatic EVs are used to predict the site of infection exacerbation and symptom development. Two isolation protocols that make use of magnetic beads conjugated to antibodies can be used. In the first, total EVs are first isolated by using beads conjugated to a pan-EV marker such as CD81, CD9 or CD63.This is followed by the isolation of tissue/cell-type specific EVs. In the second protocol, tissue/cell-type specific EV isolation is performed directly from the sample. In either protocol magnetic beads are conjugated to an antibody, or a plurality of antibodies that are specific to EVs of the desired tissue/cell-type. The beads are then incubated with the sample or the isolated total EVs and the beads are then removed by a magnet. The EVs are eluted from the beads and the pure population is analyzed.

In one example, blood from a patient suffering from COVID-19 is provided. COVID-19 produces a wide variety of symptoms, including cognitive symptoms, heart symptoms, blood vessel symptoms, skin symptoms, lung symptoms and many others. There is currently no way to predict which type of symptoms any given subject will develop nor what their severity will be. EVs from neurons, heart, blood vessels, the lungs and other tissue/cell types are isolated and analyzed for factors that indicate inflammation and damage. Sites of COVID-19 symptomatic development are thus identified.

In a similar example, a patient suffering from long COVID, a syndrome comprising long lasting symptoms after the initial infection has dissipated, provides a blood sample. EVs are isolated from tissues in which the subject is suffering symptoms and the presence of markers of damage is quantified. As the subject receives treatment, receives a vaccine or heals naturally EVs are monitored again at a later time point. The quantity of factors indicative of damage is compared to the previous measuring and improvement is identified by a decrease or disappearance of these factors.

In a further example, a patient suffering from cancer is monitored for residual disease/relapse after treatment. Following the removal of a tumor or successful treatment with an anticancer therapeutic EVs from the tissue/cell-type that previously harbored the cancer are monitored for the reappearance or residual presence of factors that indicate the return of cancer (or that it never fully was removed). These factors appear before the relapse residual disease produces adverse symptoms or can be detected by conventional means. The subject can therefore receive treatment in a more timely manner. Similarly, a patient suffering from cancer is given an anticancer and to monitor the therapeutics effect EVs are analyzed at various time points. A decrease in factors that indicate cellular damage shows that the therapeutic is effective. At the same time healthy non-cancerous tissues are monitored for off-target effects produced by the therapeutic.

### Example 3: Methods of Treating Using EVs

Once a physiopathological state is identified in cells of a patient the next step is to treat those cells. EV therapeutics offer an ideal method of treatment as they home to locations of disease, inflammation and damage.

Lung injury is often caused by a hyper pro-inflammatory response and can be detected using the method of the invention described hereinabove. This response is often commonly referred to as a “cytokine storm”, and it is highly advantageous to know which patients are likely to develop this life-threatening condition. One of the important components of the immune system that contribute to the hyperactivation of the cytokine storm is macrophages.

As demonstrated hereinbelow, it was found that chorionic (CH) and umbilical cord (UC)-derived MSCs, and extracellular vesicles (EVs) secreted by these cells, induce high levels of the IL-10 in LPS-treated macrophages and decreased the secretion of IL-6 and TNF-alpha that contribute to cytokine storm. Similarly, these two MSC subpopulations induced higher expression of VEGF and KGF.

The ability of specific MSC and EV subpopulations to exert preferential effect on macrophage activation was tested. To mimic cytokine storm, LPS-treated macrophages were employed. THP-1 (human monocytes) were stimulated with PMA 100 nM to induce macrophage differentiation. The macrophages were then either co-cultured with different MSC subpopulations in transwell plates (1 mM filters) or with purified EVs isolated from MSC cultures, and this culture was followed by stimulation with LPS (20 ng/ml). Cytokine expression was measured by RT-PCR.

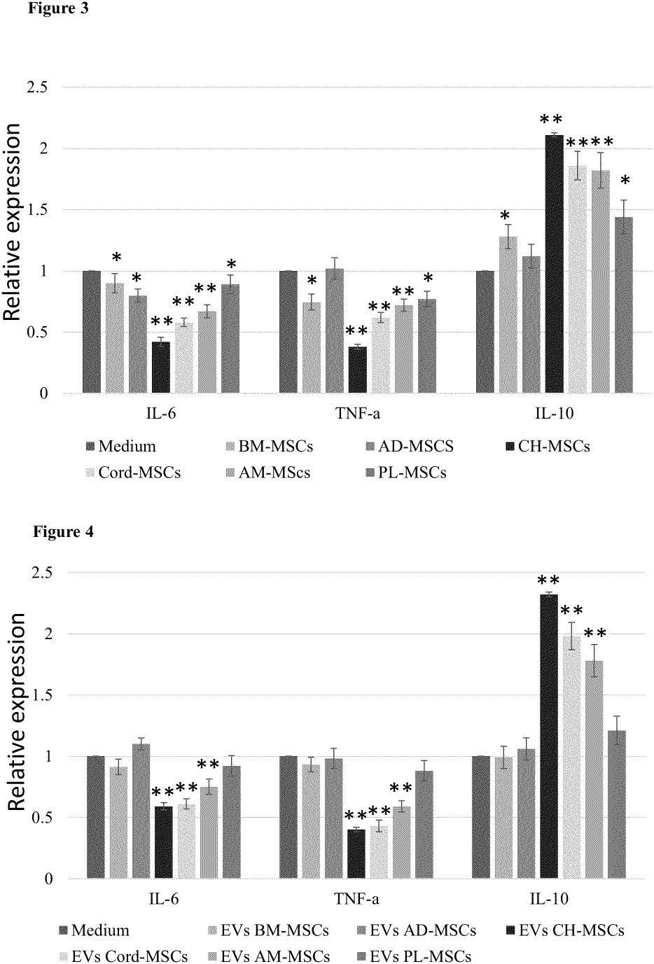

As presented in FIGS. 3 and 4, MSCs and EVs derived from chorionic placenta (CH) exerted the strongest anti-inflammatory and pro-M2 effects followed by umbilical cord (Cord) derived MSCs and EVs and then by amniotic membrane (AM) derived MSCs and EVs. Bone marrow (BM) and PL-MSCs (decidua basalis) derived cells also had an effect though it was greatly reduced, and adipose (AD) derived cells were the least effective. The extracellular vesicles (EVs) of these cells had comparable effects, with CH and Cord EVs having the greatest effect. These results demonstrate that choosing the right source of cells and EVs has important implications for the therapeutic effects of the cells on macrophages in general and cytokine storm in specific.

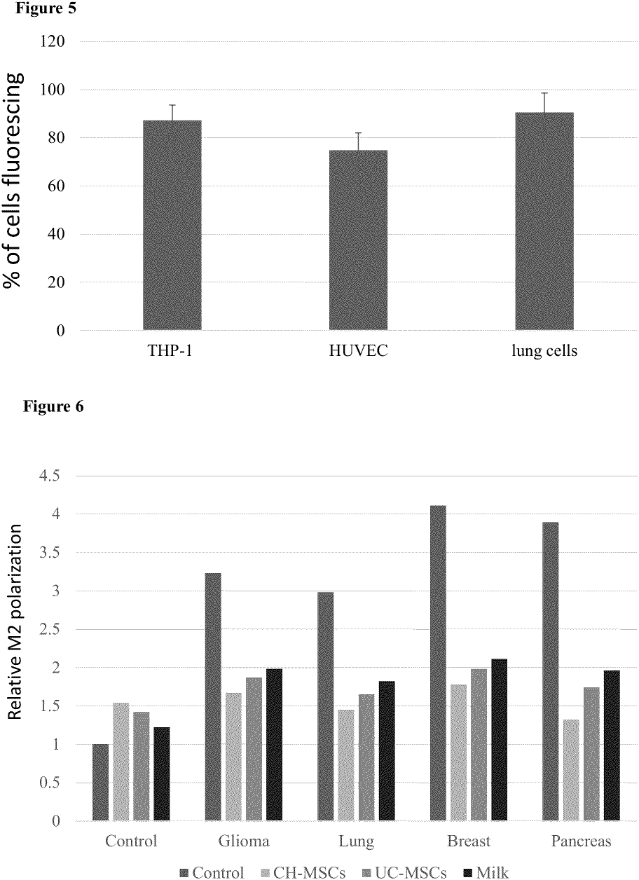

The function of the EVs is likely carried out by the delivery of cargo into the cytoplasm of target cells. By tracking fluorescent EVs from chorionic placenta MSCs it was determined that the vesicles are able to internalize their cargo into human macrophage, epithelial alveolar cells and HUVEC endothelial cells (FIG. 5).

Various combinations of miRs and anti-miRs expressed in EVs were tested for their ability to inhibit the proinflammatory response in macrophages. EVs expressing the combination of miR-124 and anti-miR-214 have the greatest potential in inhibiting pro-inflammatory response in LPS-activated THP-1 cells. These EVs inhibited the production of IL-6 by 81 % and that of TNF-alpha by 77.65%. Similarly, EVs carrying miR-145 or miR-27a and anti-miR-214 exerted a similar effect. In contrast, the combination of miR-155 and anti-miR-214 did not reduce the expression of TNF-alpha and IL-6 but rather increased their expression by 19 and 22 % respectively.

Further, it is shown that miRs inhibiting the NF-KB pathway include any one of miR-20, miR-9, miR-506, miR-124 or miR-455 in combination with miR-21 and miR-155 inhibit pro-inflammatory responses.

This Example shows the various inflammatory disorders associated with hyperactivation of the immune system, including but not limited to ARDS, sepsis, and cytokine storm, can be treated using subpopulations of MSCs and EVs.

Cord and placenta derived MSCs, specifically chorionic MSCs and/or their EVs may be administered locally, intravenously, intranasally, or by inhalation and decrease lung damage, inflammatory response, promote angiogenesis and regeneration.

### Example 4: Methods of Treating Cancer EVs

Cancerous cells are known to produce EVs with pro-tumorigenic cargo and that exert pro-tumorigenic effects. This was demonstrated in a human macrophage system. Human THP-1 cells were differentiated into macrophages using 10 nM PMA. The differentiated macrophages were then treated with EVs isolated from glioma, lung, breast and pancreas tumor cell lines. The conversion of the macrophages to pro-tumorigenic M2 macrophages was monitored by the expression of the M2 marker CD206, as measured by RT-PCR. All the tumor cell line derived EVs increased the conversion to M2 macrophages highlighting the universality of this trait of cancer EVs (FIG. 6). EVs stained with the fluorescent dye CellTracker Red were also used and it was observed that the tumor EVs entered the macrophages in high numbers.

However, when the macrophages were incubated with tumor EVs in the presence of EVs secreted from chorionic placenta (CH) or umbilical cord (UC) MSCs or milk-derived EVs the pro-tumorigenic effects were largely abrogated (FIG. 6). It was found that the addition of MSC and milk-derived EVs nearly completely reversed the increased M2 polarization of THP-1 cells. Interestingly, CH-MSC derived EVs were consistently superior to UC-MSCs EVs, which were themselves consistently superior to milk-derived EVs. Further, the addition of the MSC and milk-derived EVs significantly decreased the entrance of the labeled tumor EVs into the cells. These results indicate that addition of EVs with an anti-tumor capability can compete with tumor EVs and largely block there pro-tumor forming effects. This indicates not only a profound usefulness in treating cancer, but in halting metastasis as the tumor EVs may prime a metastatic location for tumor development. This is especially of use when the diagnostic method of the invention has determined the presence of residual disease, disease relapse or early metastasis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of detecting an adverse cellular state in a subject in need thereof, the method comprising:
(i) providing a sample obtained from said subject, said sample comprising extracellular vesicles (EVs);
(ii) isolating from said sample a population of EVs originating from a tissue or cell type; and
(iii) determining in said population of EVs a presence of at least one factor indicative of said adverse cellular state;
wherein said presence of said at least one factor indicates said adverse cellular state is present in said tissue or cell type;
thereby detecting an adverse cellular state in a subject in need thereof.

2. The method of claim 1, wherein said isolating comprises determining whether an EV of said population comprises a tissue, or cell-type specific marker; and wherein a positive determination of a presence of said tissue, or cell-type specific marker is indicative of the origin of said EV.

3. The method of claim 1, wherein said isolating comprises contacting said sample with an antibody or antigen binding fragment specific to a tissue or cell-type specific marker and isolating said antibody or antigen binding fragment.

4. The method of claim 3, wherein said antibody or antigen binding fragment is conjugated to a solid support and said isolating comprises isolating said solid support.

5. The method of claim 4, wherein said solid support is a magnetic bead.

6. The method of claim 2, wherein said tissue or cell-type specific marker is a tissue or cell-type specific surface protein.

7. The method of claim 6, wherein said tissue or cell-type specific surface protein is selected from Table 1.

8. The method of claim 1, further comprising before (ii) isolating total EVs from said sample and wherein said population of EVs is isolated from said total EVs.

9. The method of claim 8, wherein said total EVs are isolated by contacting said sample with an antibody or antigen binding fragment specific to EVs and isolating said antibody or antigen binding fragment.

10. The method of claim 9 wherein said antibody or antigen binding fragment specific to EVs a) is conjugated to a solid support, b) binds to a target selected from CD81, CD9 and CD63 or c) both.

11. The method of claim 1, wherein said adverse cellular state is selected from an inflammatory state, a damaged state, a hypoxic state, a stressed state, a state of infection, an immunosuppressed state, a metastatic state and a hyper-proliferative state.

12. The method of claim 11, wherein said at least one factor indicative of said adverse cellular state is selected from a protein and a regulatory RNA.

13. The method of claim 12, wherein said regulatory RNA is selected from a microRNA (miR), a long noncoding RNA (lncRNA) and a circular RNA (circRNA).

14. The method of claim 12, wherein said regulatory RNA is selected from Table 2.

15. The method of claim 1, wherein said adverse cellular state is indicative of a pathological condition in said subject.

16. The method of claim 1 , wherein said subject is selected from: (i) a subject who suffers from a disease and said cellular state is indicative of disease activity in said tissue or cell-type, disease symptoms in said tissue or cell-type, disease origin from said tissue or cell-type or a combination thereof; and (ii) and a subject who suffers from a disease and is receiving treatment for said disease and wherein said method is a method of determining responsiveness of said subject to said treatment.

17. The method of claim 1 , wherein said method is a method of early detection of a disease or of a disease symptom.

18. (canceled)

19. The method of claim 1, wherein a decrease in said adverse cellular state in said subject as compared to a time point before said treatment or earlier in said treatment indicates responsiveness to said treatment.

20. The method of claim 1, further comprising administering a treatment to said subject wherein said treatment is selected to treat said adverse cellular condition, to treat said tissue or cell type or both.

21. The method of claim 20, wherein said treatment comprises therapeutic extracellular vesicles, optionally wherein said therapeutic extracellular vesicles are selected from extracellular vesicles derived from any one of mesenchymal stromal cells (MSCs), plant cells and milk, further optionally wherein said extracellular vesicles are derived from chorionic placenta MSCs, umbilical cord MSCs or milk.

22. (canceled)

23. (canceled)
